(19) United States
(12) Patent Application Publication
(10) Pub. No.: US 2022/0378292 A1
(43) Pub. Date: Dec. 1, 2022

(54) ELECTRICAL ACTIVITY-BASED PROCEDURE GUIDANCE

(71) Applicant: Navix International Limited, Rroad Town (VG)

(72) Inventor: Shlomo BEN-HAIM, Milan (IT)

(73) Assignee: Navix International Limited, Road Town (VG)

(21) Appl. No.: 17/762,794

(22) PCT Filed: Sep. 24, 2020
(86) PCT No.: PCT/IB2020/058952

§ 371 Date: Mar. 23, 2022

Related U.S. Application Data
(60) Provisional application No. 62/978,894, filed on Feb. 20, 2020.
(60) Provisional application No. 62/927,712, filed on Oct. 30, 2019.
(60) Provisional application No. 62/956,253, filed on Jan. 1, 2020.
(60) Provisional application No. 62/911,294, filed on Oct. 6, 2019.
(60) Provisional application No. 62/905,554, filed on Sep. 25, 2019.

(51) Int. Cl. A61B 5/00; A61B 5/0538

(52) U.S. Cl. CPC A61B 5/0036; A61B 5/0044; A61B 5/0538; A61B 5/0035

(57) ABSTRACT

Heart tissue electrical activity mapping used to guide the placement of devices to intervene in (treat) structural heart disease. In some embodiments, the intervention comprises placement of an implantable device, and/or positioning of a therapeutic device used to remove and/or remodel tissue. In some embodiments, electrical activity mapping is performed along with spatial mapping of a body cavity. In some embodiments, the intervention device position is compared to the measured positions of anatomical structures critical to heart electrical function to assess and/or prevent complications due to the device damaging heart electrical function.

### RELATED APPLICATIONS

This application claims the benefit of priority under 35 USC § 119(e) of:
U.S. Provisional Patent Application No. 62/905,554 filed Sep. 25, 2019;
U.S. Provisional Patent Application No. 62/911,294 filed Oct. 6, 2019;
U.S. Provisional Patent Application No. 62/956,253 filed Jan. 1, 2020; and
U.S. Provisional Patent Application No. 62/927,712 filed Oct. 30, 2019;

the contents of each of which are incorporated herein by reference in their entirety.

### FIELD AND BACKGROUND OF THE INVENTION

The present invention, in some embodiments thereof, relates to the field of navigation within body cavities by intrabody devices, and more particularly, to guidance of the placement of intrabody devices, optionally including implantable devices.

Several medical procedures in cardiology and other medical fields comprise the use of intrabody devices such as catheter probes to reach tissue targeted for diagnosis and/or treatment while minimizing procedure invasiveness. Imaging-based techniques (including, for example, real time fluoroscopy, angiography, transthoracic ultrasound, trans-esophageal ultrasound, intracardiac ultrasound, as well as information pre-acquired from CT or MRI) for navigation of the catheter and monitoring of treatments continue to be refined, and are now joined by techniques and systems such as the use of electrical field measurement-guided position sensing systems.

A variety of catheter-delivered intrabody devices are in current use for purposes of treatment and/or diagnosis, including implantable pacemakers, stents, implantable rings, implantable valve replacements (such as: aortic valve replacement, mitral valve replacement and tricuspid valve replacement), left atrial appendage (LAA) occluders, and/or atrial septal defect (ASD) occluders.

### SUMMARY OF THE INVENTION

According to an aspect of some embodiments of the present disclosure, there is provided a method of guiding a procedure for implanting a structural heart disease intervention device, the method including: accessing a structural representation of a portion of a heart; accessing electrophysiological measurements indicating electrical activity of tissue of the heart; accessing position measurements indicating a position of the device within the portion of the heart; processing the electrophysiological measurements to identify a location of a heart structure within the portion of the heart; and presenting the heart structure location, relative to the position of the device; wherein the electrophysiological measurements and the position measurements are obtained during the procedure being guided.

According to some embodiments of the present disclosure, the presenting includes presenting a display showing the portion of the heart, a position of the device within the portion of the heart, and an indication of the position of the heart structure within the portion of the heart.

According to some embodiments of the present disclosure, the processing includes production of a structural model of the portion of the heart wherein the heart structure location is represented as a characteristic of a portion of the structural model.

According to some embodiments of the present disclosure, the presenting including presenting a warning that the heart structure location is in a dangerous proximity to the device.

According to some embodiments of the present disclosure, the warning instructs not performing a procedure action.

According to some embodiments of the present disclosure, the presenting including presenting a confirmation that the heart structure location is at a sufficient distance from the device.

According to some embodiments of the present disclosure, the confirmation instructs a procedure action is allowed.

According to some embodiments of the present disclosure, the presenting including presenting a confirmation that the heart structure location is in a sufficient proximity to the device.

According to some embodiments of the present disclosure, the confirmation instructs performing a procedure action.

According to some embodiments of the present disclosure, the presenting including presenting a warning that the heart structure location is in insufficient proximity to the device.

According to some embodiments of the present disclosure, the warning instructs not performing a procedure action.

According to some embodiments of the present disclosure, the procedure action includes at least one from the group consisting of: attaching the device expanding the device, contracting the device, retrieving the device, at least partially de-implanting the device, penetrating tissue with the device, pacing heart electrical activity using the device, and measuring heart electrical activity using the device.

According to some embodiments of the present disclosure, the structural heart disease intervention device is a device selected from the group consisting of: a left atrial appendage occluder, a transcatheter aortic valve implant, a mitral valve clip, a tricuspid valve clip, a mitral annuloplasty ring or band, a tricuspid annuloplasty ring or band, and an atrial septal defect closure device.

According to some embodiments of the present disclosure, the position measurements indicating the position of the device comprise electrical field measurements made using an electrode of the device.

According to some embodiments of the present disclosure, the position measurements indicating the position of the device comprise EP measurements made using an electrode of the device.

According to some embodiments of the present disclosure, the EP measurements are made using an electrode of the device.

According to some embodiments of the present disclosure, the EP measurements are made using an electrode of the device to pace the electrical activity of tissue of the heart.

According to some embodiments of the present disclosure, the structural representation is a three-dimensional structural representation.

According to an aspect of some embodiments of the present disclosure, there is provided a method of guiding a procedure for implanting a structural heart disease intervention device in a heart, the method including: accessing a baseline value of a parameter of an EP signal measurement; accessing EP signal measurements obtained after an action performed using the device, the action being performed upon a heart wall and/or a heart valvular apparatus; processing, using a computer processor, the EP signal measurements to identify a change of the parameter from the baseline value; and presenting guidance for a next action to be performed, based on the identified change.

According to some embodiments of the present disclosure, the action affects a mechanical and/or hemodynamic function of the heart.

According to some embodiments of the present disclosure, the processing includes estimating a location of the heart having changed electrophysiological activity resulting in the identified change.

According to some embodiments of the present disclosure, the presenting includes displaying a structural representation of the heart including an indication of the location of the heart having changed electrophysiological activity resulting in the identified change.

According to some embodiments of the present disclosure, the identified change includes a change in at least one of the group consisting of: a waveform morphology, a waveform amplitude, a waveform duration, a waveform frequency, a waveform slope, and a spectral content of an EP signal.

According to some embodiments of the present disclosure, the changed parameter includes at least one of the group consisting of: EP signal rate, EP signal rhythm, variability in EP signal rhythm, QRS duration, QRS spectral content, P wave morphology, PR interval, ST segment, and T wave morphology.

According to some embodiments of the present disclosure, the changed parameter includes a conduction time measured for at least one of the group consisting of conduction time between: electrodes, locations, EP signal components, and delivery of a pacing signal and a signal arrival at a location.

According to some embodiments of the present disclosure, the changed parameter includes an intracardially measured local activation time of a first location within the heart, relative to at least one of the group consisting of: a phase of a BS-ECG, a local activation time of a second location within the heart measured intracardially, and a previously measured intracardially measured local activation time of the first location within the heart.

According to some embodiments of the present disclosure, the processing includes determination of a change in a result of a pacing test, or a change in a measurement value with and without pacing.

According to some embodiments of the present disclosure, the identified change indicates potential damage to a heart electrical system structure, and the guidance includes instruction to reverse an implantation of the structural heart disease intervention device.

According to some embodiments of the present disclosure, the identified change indicates a level of isolation between components of the heart electrical system, and the guidance includes an indication as to whether a targeted level of isolation has been achieved.

According to some embodiments of the present disclosure, the accessed electrophysiological measurements indicating electrical activity of tissue of the heart comprise measurements of an electrophysiological study described by a CPT code.

According to an aspect of some embodiments of the present disclosure, there is provided a system including a processor, memory, and display, wherein the memory is configured with instructions instructing the processor to: access a baseline value of a parameter of an EP signal measurement; access EP signal measurements obtained after an action performed within the heart which modifies at least a mechanical and/or hemodynamic function of the heart; process the EP signal measurements to identify a change of the parameter from the baseline value; and present, using the display, guidance for a next action to be performed within the heart, wherein the guidance presented is based on the identified change.

According to some embodiments of the present disclosure, the EP signal measurements are obtained using an electrode with the heart, and the guidance guides a next action performed using a structural heart disease treatment device positioned within the heart.

According to some embodiments of the present disclosure, the processor is further instructed to estimate a position and/or identity of a location of the heart having changed electrophysiological activity resulting in the identified change.

According to some embodiments of the present disclosure, guidance presented includes a structural representation of the heart including an indication of a position and/or identity of a location of the heart having changed electrophysiological activity resulting in the identified change.

According to some embodiments of the present disclosure, the processor is instructed to identify a change in at least one of the group consisting of: an EP signal waveform morphology, an EP signal waveform amplitude, an EP signal waveform duration, an EP signal waveform frequency, an EP signal waveform slope, and a spectral content of an EP signal.

According to some embodiments of the present disclosure, the changed parameter includes at least one of the group consisting of: EP signal rate, EP signal rhythm, variability in EP signal rhythm, QRS duration, QRS spectral content, P wave morphology, PR interval, ST segment, and T wave morphology.

According to some embodiments of the present disclosure, the changed parameter includes a conduction time measured for at least one of the group consisting of conduction time between: electrodes, locations, EP signal components from signal sources located along a transmission pathway, and delivery of a pacing signal and a signal arrival at a location.

According to some embodiments of the present disclosure, the changed parameter includes an intracardially measured local activation time of a first location within the heart, relative to at least one of the group consisting of: a phase of a BS-ECG, a local activation time of a second location within the heart measured intracardially, and a previously measured intracardially measured local activation time of the first location within the heart.

According to some embodiments of the present disclosure, the EP signal measurements are processed to determine a change in a result of a pacing test, or a change in a measurement value with and without pacing.

According to some embodiments of the present disclosure, the identified change indicates potential damage to a heart electrical system structure, and the guidance includes instruction to reverse an implantation of the structural heart disease intervention device.

According to some embodiments of the present disclosure, the identified change indicates a level of isolation between components of the heart electrical system, and the guidance includes an indication as to whether a targeted level of isolation has been achieved.

According to an aspect of some embodiments of the present disclosure, there is provided a method of guiding a procedure for implanting a structural heart disease intervention device in a heart, the method including: accessing reference EP signal measurements; accessing identifying EP signal measurements including a signal component characteristic of a location within the heart; processing, using a computer processor, the reference and identifying EP signal measurements to identify the location, based on timing of an event in the identifying EP signal measurements, relative to events in the reference EP signal measurements; and presenting guidance for a next action to be performed, based on the identified location.

According to some embodiments of the present disclosure, the identifying EP signal measurements comprise an IC-ECG measured at a first location within the heart, and the reference EP signal measurements comprise an IC-ECG measured at a second location within the heart.

According to some embodiments of the present disclosure, the reference EP signal measurements comprise a BS-ECG, and the identifying EP signal measurements includes an IC-ECG.

According to some embodiments of the present disclosure, the processing includes identifying a source location of an EP signal measured by the identifying EP signal measurements as including a first particular heart electrical system structure, based on comparison to reference EP signal measurements including at least one of the group consisting of: an electrogram associated with a second particular heart electrical system structure, and a previously measured electrogram associated with the first particular heart electrical system structure.

According to some embodiments of the present disclosure, the accessed EP signal measurements comprise measurements of an electrophysiological study described by a CPT code.

According to an aspect of some embodiments of the present disclosure, there is provided a system including a processor, software, and display, wherein the memory is configured with instructions instructing the processor to: access reference EP signal measurements; access identifying EP signal measurements characteristic of a location within a heart; process the reference and identifying EP signal measurements to identify the location, based on timing of an event in the identifying EP signal measurements, relative to events in the reference EP signal measurements; and present guidance for a next action to be performed, based on the identified location.

According to some embodiments of the present disclosure, the identifying EP signal measurements comprise an IC-ECG measured at a first location within the heart, and the reference EP signal measurements comprise an IC-ECG measured at a second location within the heart.

According to some embodiments of the present disclosure, the reference EP signal measurements comprise a BS-ECG, and the identifying EP signal measurements comprise an IC-ECG.

According to some embodiments of the present disclosure, the processor is instructed to identify a source location of an EP signal measured by the identifying EP signal measurements including a first particular heart electrical system structure, based on comparison to reference EP signal measurements including at least one of the group consisting of: an electrogram associated with a second particular heart electrical system structure, and a previously measured electrogram associated with the first particular heart electrical system structure.

According to an aspect of some embodiments of the present disclosure, there is provided a method of guiding a medical procedure within a body cavity including: accessing a rule, wherein the rule maps electrical field measurements to body cavity positions; accessing measurements of heart electrical function, the measurements being associated to the body cavity positions; accessing data indicative of a position within the body cavity of a device used to provide structural heart disease intervention therapy; using the rule, the measurements of heart electrical function, and the position to produce an image and/or model indicating a feature of heart electrical functioning at the position; and providing guidance indicating device position relative to features of the image and/or model, including in relation to one or more signal sources of the measurements of heart electrical function.

According to some embodiments of the present disclosure, the measurements of heart electrical function are associated to the body cavity positions by measurement from a same probe at a same position of the probe.

According to some embodiments of the present disclosure, the measurements of heart electrical function are associated to the body cavity positions by measurement from a same probe during a same time.

According to some embodiments of the present disclosure, the measurements of heart electrical function indicate activity of a bundle of His.

According to some embodiments of the present disclosure, the measurements of heart electrical function indicate activity of an AV node.

According to some embodiments of the present disclosure, the measurements of heart electrical function indicate activity of a phrenic nerve.

According to some embodiments of the present disclosure, the device includes one of: an annuloplasty ring; a heart valve clip; a left atrial appendage occluder; a ventricular septal defect clip; a heart valve implant; and a heart valve replacement.

According to some embodiments of the present disclosure, the position of the device is measured using the device as an electrode.

According to some embodiments of the present disclosure, the position of the device is measured using an electrode probe in contact with the device.

According to some embodiments of the present disclosure, the measurements of heart electrical function are made along with measurement of a reference signal, and the using includes comparing the reference signal to the heart electrical signal measurements.

According to some embodiments of the present disclosure, the accessed electrophysiological measurements indicating electrical activity of tissue of the heart comprise measurements of an electrophysiological study described by a CPT code.

According to an aspect of some embodiments of the present disclosure, there is provided a system including a processor, memory, and display, wherein the memory is configured with instructions instructing the processor to: access a rule, wherein the rule maps electrical field measurements to body cavity positions; access measurements of heart electrical function, the measurements being associated to the body cavity positions; access data indicative of a position within the body cavity of a device used to provide structural heart disease intervention therapy; use the rule, the measurements of heart electrical function, and the position to produce an image and/or model indicating a feature of heart electrical functioning at the position; and provide, using the display, guidance indicating device position relative to features of the image and/or model, including in relation to one or more signal sources of the measurements of heart electrical function.

According to some embodiments of the present disclosure, the measurements of heart electrical function are associated to the body cavity positions by measurement from a same probe at a same position of the probe.

According to some embodiments of the present disclosure, the measurements of heart electrical function are associated to the body cavity positions by measurement from a same probe during a same time.

According to some embodiments of the present disclosure, the device includes one of: an annuloplasty ring; a heart valve clip; a left atrial appendage occluder; a ventricular septal defect clip; a heart valve implant; and a heart valve replacement.

According to some embodiments of the present disclosure, the measurements of heart electrical function are made along with measurement of a reference signal, and processor compares the reference signal to the heart electrical signal measurements to produce the image and/or model.

According to an aspect of some embodiments of the present disclosure, there is provided a method of guiding a medical procedure within a body cavity, including: accessing measurements of heart electrical function, the measurements being associated to positions within the body cavity; accessing data indicative of a position within the body cavity of a device used to provide structural heart disease intervention therapy; using the measurements of heart electrical function, their positions, and the position of the device to determine whether the device and one or more signal sources of the measurements of heart electrical function coincide; and providing guidance indicating device position in relation to the one or more signal sources of the measured heart electrical function.

According to some embodiments of the present disclosure, the heart electrical field measurements are associated to the body cavity positions by measurement from a same probe at a same position of the probe.

According to an aspect of some embodiments of the present disclosure, there is provided a method of guiding a procedure to intervene in heart structural disease, including: selecting to do a procedure to intervene in a heart structural disease, wherein the procedure is associated with a potential complication including damage to a critical structural feature of heart electrical function; mapping electrical function in at least the critical structural feature using a same tool used to guide positioning within the procedure to produce a map of electrical function; and performing the procedure using the tool and the map of electrical function.

According to an aspect of some embodiments of the present disclosure, there is provided a method of indicating positional relationship between an identified heart tissue and a device configured to provide structural heart disease intervention therapy, the method including: accessing measurements, made by an in-heart electrode, and indicative of electrical activity of heart tissue in the vicinity of the in-heart electrode; identifying the heart tissue based on the measurements; identifying a position of the heart tissue based on data indicative of the position of the in-heart electrode when the in-heart electrode measured the electrical activity of the heart tissue; accessing measurements indicative of a position of the device; and indicating, on an image of the heart, the position identified for the heart tissue, the identification of the heart tissue, and the position of the device.

According to some embodiments of the present disclosure, the heart tissue is identified as a tissue that should not be intervened with by the device.

According to some embodiments of the present disclosure, the heart tissue is identified as the AV node.

According to some embodiments of the present disclosure, the heart tissue is identified as the bundle of His.

According to some embodiments of the present disclosure, the heart tissue is identified as one of an artery or a ventricle.

According to some embodiments of the present disclosure, the heart tissue is identified based on comparison of ECG signals measured by the in-heart electrode during some time period, and surface ECG signals measured during the same time period.

According to some embodiments of the present disclosure, the device includes one of: an annuloplasty ring; a heart valve clip; a left atrial appendage occluder; a ventricular septal defect clip; a heart valve implant; and a heart valve replacement.

According to some embodiments of the present disclosure, the position of the device is measured using the device or a portion thereof as an electrode.

According to some embodiments of the present disclosure, the position of the device is measured using an electrode probe in contact with the device.

According to some embodiments of the present disclosure, the measurements made by the in-heart electrode are measurements of intracardiac electrogram data.

According to some embodiments of the present disclosure, indicating on the image the identification of the heart tissue includes coloring the position of the heart tissue in the image by a color predetermined for that heart tissue.

According to an aspect of some embodiments of the present disclosure, there is provided a method of guiding a structural heart disease intervention, including: accessing a structural representation of a heart; accessing electrophysiological measurements indicating electrical activity of tissue of the heart; associating the electrophysiological measurements to locations in the structural representation of the heart corresponding to locations at which the measurements were recorded; selecting a location for attachment of a device configured to provide structural heart disease intervention, based on the structural representation, the electrophysiological measurements, and their locations in the structural representation; and presenting an image of the structural representation of the heart wherein the selected location is marked.

According to an aspect of some embodiments of the present disclosure, there is provided a method of guiding a structural heart disease intervention, including: accessing electrophysiological (EP) measurements of the heart measured from a specified location; and guiding the structural heart disease intervention based on the accessed EP measurements.

According to some embodiments of the present disclosure, guiding the structural heart disease intervention includes indicating on an image of a portion of the heart a current location of an implant for use in the intervention, the specified location, and the accessed EP measurements.

According to some embodiments of the present disclosure, guiding the structural heart disease intervention includes guiding an intervention tool to a location within the heart to be treated with the structural heart disease intervention.

Unless otherwise defined, all technical and/or scientific terms used herein have the same meaning as commonly understood by one of ordinary skill in the art to which the present disclosure pertains. Although methods and materials similar or equivalent to those described herein can be used in the practice or testing of embodiments of the present disclosure, exemplary methods and/or materials are described below. In case of conflict, the patent specification, including definitions, will control. In addition, the materials, methods, and examples are illustrative only and are not intended to be necessarily limiting.

As will be appreciated by one skilled in the art, aspects of the present disclosure may be embodied as a system, method or computer program product. Accordingly, aspects of the present disclosure may take the form of an entirely hardware embodiment, an entirely software embodiment (including firmware, resident software, microcode, etc.) or an embodiment combining software and hardware aspects that may all generally be referred to herein as a “circuit,” “module” or “system” (e.g., a method may be implemented using “computer circuitry”). Furthermore, some embodiments of the present disclosure may take the form of a computer program product embodied in one or more computer readable medium(s) having computer readable program code embodied thereon. Implementation of the method and/or system of some embodiments of the present disclosure can involve performing and/or completing selected tasks manually, automatically, or a combination thereof. Moreover, according to actual instrumentation and equipment of some embodiments of the method and/or system of the present disclosure, several selected tasks could be implemented by hardware, by software or by firmware and/or by a combination thereof, e.g., using an operating system.

For example, hardware for performing selected tasks according to some embodiments of the present disclosure could be implemented as a chip or a circuit. As software, selected tasks according to some embodiments of the present disclosure could be implemented as a plurality of software instructions being executed by a computer using any suitable operating system. In some embodiments of the present disclosure, one or more tasks performed in method and/or by system are performed by a data processor (also referred to herein as a “digital processor”, in reference to data processors which operate using groups of digital bits), such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well. Any of these implementations are referred to herein more generally as instances of computer circuitry.

Any combination of one or more computer readable medium(s) may be utilized for some embodiments of the present disclosure. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device. A computer readable storage medium may also contain or store information for use by such a program, for example, data structured in the way it is recorded by the computer readable storage medium so that a computer program can access it as, for example, one or more tables, lists, arrays, data trees, and/or another data structure. Herein a computer readable storage medium which records data in a form retrievable as groups of digital bits is also referred to as a digital memory. It should be understood that a computer readable storage medium, in some embodiments, is optionally also used as a computer writable storage medium, in the case of a computer readable storage medium which is not read-only in nature, and/or in a read-only state.

Herein, a data processor is said to be “configured” to perform data processing actions insofar as it is coupled to a computer readable memory to receive instructions and/or data therefrom, process them, and/or store processing results in the same or another computer readable storage memory. The processing performed (optionally on the data) is specified by the instructions. The act of processing may be referred to additionally or alternatively by one or more other terms; for example: comparing, estimating, determining, calculating, identifying, associating, storing, analyzing, selecting, and/or transforming. For example, in some embodiments, a digital processor receives instructions and data from a digital memory, processes the data according to the instructions, and/or stores processing results in the digital memory. In some embodiments, “providing” processing results comprises one or more of transmitting, storing and/or presenting processing results. Presenting optionally comprises showing on a display, indicating by sound, printing on a printout, or otherwise giving results in a form accessible to human sensory capabilities.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present disclosure may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present disclosure may be described below with reference to flowchart illustrations and/or block diagrams of methods, apparatus (systems) and computer program products according to embodiments of the present disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks

### BRIEF DESCRIPTION OF THE SEVERAL VIEWS OF THE DRAWINGS

Some embodiments of the present disclosure are herein described, by way of example only, with reference to the accompanying drawings. With specific reference now to the drawings in detail, it is stressed that the particulars shown are by way of example, and for purposes of illustrative discussion of embodiments of the present disclosure. In this regard, the description taken with the drawings makes apparent to those skilled in the art how embodiments of the present disclosure may be practiced.

In the drawings:

FIG. 1A is a flowchart schematically representing a method of producing computer-generated guidance for a structural heart disease treatment (e.g., treatment via MISHDI), according to some embodiments of the present disclosure;

FIG. 1B is a flowchart schematically representing a method of producing guidance for a structural heart disease treatment (e.g., via MISHDI), according to some embodiments of the present disclosure;

FIG. 1C is a flowchart schematically representing a method of using rule-generated guidance for a structural heart disease treatment (e.g., via MISHDI) to produce a guidance image, according to some embodiments of the present disclosure;

FIG. 1D is a flowchart schematically representing a method of guiding a structural heart disease treatment (e.g., via MISHDI), according to some embodiments of the present disclosure;

FIG. 2 schematically represents implantation of an annuloplasty ring around a tricuspid valve between a right atrium and a right ventricle of a heart, according to some embodiments of the present disclosure;

FIG. 3 is a flowchart of a method for using EP signals in the identification of tissue near a measurement device, according to some embodiments of the present disclosure; and

FIG. 4 schematically represents a block diagram of a system configured to use a compound model incorporating EP signal data to guide SHD interventions, according to some embodiments of the present disclosure.

### DESCRIPTION OF SPECIFIC EMBODIMENTS OF THE INVENTION

The present invention, in some embodiments thereof, relates to the field of navigation within body cavities by intrabody devices, and more particularly, to guidance of the placement of intrabody devices, optionally including implantable devices.

### Overview

A broad aspect of some embodiments of the present disclosure relates to mapping two or more measured aspects of organ function to a unified display of the organ. More particularly, in some embodiments, heart electrophysiological (EP) measurements (of electrophysiological function) are mapped to a structural display of the heart. In some embodiments, the display is performed to assist a minimally invasive structural heart disease intervention (MISHDI), which may comprise implantation or other site-specific use and/or deployment of a device (an intervention tool). In some embodiments, a MISHDI delivers an action on one of the heart walls (pericardium, epicardium, myocardium and the septum) and/or its valvular apparatus (aortic, pulmonic, mitral and tricuspid). The action may, for example, affect mechanical and/or hemodynamic functions of the heart.

A common characteristic of MISHDI procedures is that they are performed within substantially closed-in spaces of the body, so that problems of procedure visualization and/or confirmation of intervention results potentially arise. Single measurement-mode methods used for such purposes during MISHDI—for example, for procedure visualization and/or result confirmation—have the potential drawback of being detached, when presented, from an overall context which potentially would influence how the single measurement-mode should be understood. Data from additional measurement modes may provide such context, but integrating results of different measurement modes may add to the already heavy cognitive demands from the physician carrying out the intervention. A unified display potentially provides context, while reducing reliance on a clinician's ability to mentally integrate disparate inputs from a plurality of different measurement modes.

The unified display is configured, in some embodiments, to support navigation and/or deployment of the implanted device according to particulars of one or both of heart structure and heart function; for example, to provide guidance allowing deploying and/or attaching the device where it performs its function as a structural heart disease (SHD) treatment (herein, devices as a class which directly provide MISHDI treatment are also referred to herein as “SHD intervention devices”).

Additionally or alternatively, in some embodiments, EP measurements are used to guide an SHD intervention device and/or confirm positioning of an SHD intervention device at a correct structural location, based on localizing EP measurement values and/or characteristics to certain structurally defined locations of the heart.

The normal function of the heart is based on the delicate interaction between its mechanical, electrical, neural and valvular components (“functional components”). The interaction between these components is multi-directional (e.g. electrical activity affects structure, valvular function affects electrical activity), and potentially non-linear. For example, a small anatomical disruption of the bundle of His can induce heart block that completely disrupts the heart function. Modulation of the mechanical function of a valve potentially changes stress and/or load on some or all the myocardium, potentially leading to anatomical remodeling of the myocardium. Additionally or alternatively, remodeling may be induced by the electrical activation sequence of the myocardium. Interaction with the neural system (autonomic neural system) in one or both of its afferent and efferent arms may induce a reflex change in sympathetic and/or parasympathetic tone that in return changes mechanics, hemodynamics and/or electrical properties of the heart. Furthermore, the myocardium is supplied by the coronary arteries and the venous drainage is mainly through the coronary sinus. An interruption to any of these critical structures can affect the mechanics, hemodynamics and electrical properties of the heart.

At least insofar as the heart is an intricate 3-D structure of these interacting heart functional components, the ability to perform a manipulation targeted at any one or more of these functional components while tracking potential effects on other functional components provides a potential advantage for acute and/or long term success of minimally invasive structural heart disease intervention (MISHDI). In particular, manipulations primarily targeted at one functional component may have side-effects on nearby tissue that supports a different functional component, potentially leading to unintentionally degraded function. In the case of MISHDI procedures aimed at treating structural heart disease, measurements of heart electrical function are potentially useful for guiding the procedure; for example to avoid damage from and/or monitor intended effects of an implanted SHD intervention device. More particularly, in some embodiments, measurements of EP signals are used to help identify and/or characterize locations to which an SHD intervention device is to be implanted.

A particular linkage may be noted between SHD interventions and potential risk to EP function during the performing of SHD interventions. SHD interventions can be rather extensive in contacts made with tissue (e.g., by implanted devices), exposing large areas to a risk of injury incidental to the procedure. At the same time, aspects of EP function rely on maintaining the health of relatively small and potentially delicate tissue areas with critical roles in maintaining overall heart function. Accordingly, there is a potential problem in how to efficiently perform gross structural manipulations to treat a heart while maintaining a low risk of incidental damage to the heart's electrophysiology and its specialized supporting structures.

It is noted that integration into a procedure of disparate data sources and indications of corresponding functional component states can be a complex process, potentially involving a plurality of imaging and/or sensing methodologies, each contributing part of the information needed to guide and/or confirm a minimally invasive structural heart disease intervention. Integration of information may be performed in the mind of the operator without production of a unified display. However, it is a potential advantage for information to be display-integrated, potentially reducing a risk of error or confusion, and/or reducing cognitive load on the operator. Display integration, in some embodiments, is performed by a data-integrating and data-displaying system which has been configured to connect sources of MISHDI-related data together during a procedure, by “procedure-aware” data analysis that converts data inputs, by means of processing rules, into outputs that provide procedure action-specific guidance (i.e., guidance of “guided actions”). Particularly, in some embodiments, the sources of MISHDI-related data include heart electrophysiology data.

For purposes of the descriptions herein, “display” includes visual display, and optionally or alternatively other human-sensed stimuli such as auditory and/or tactile stimuli. A “unified” display is optionally distinguished by display of action guidance arrived at through processing of measurements made by a plurality of measurement modalities. The action guidance is optionally in the form of a directive to perform an action (e.g., “do X next”/“implant now”), caution against performing an action (e.g., “don't implant now”), confirmation that an action is performed correctly (e.g., “SHD intervention device is in correct contact with tissue”), and/or an indication that an action needs to be reversed and/or mitigated (e.g., “SHD intervention device is causing a degree of EP block”).

Additionally or alternatively, action guidance includes indications positioned within an image representation of the procedure environment (e.g., a 3-D model of a heart interior). The indication may indicate location, size, and/or shape of a structure or tissue portion significant for carrying out the procedure. For example, in some embodiments, a location is identified as a valve annulus by combining structural imaging with EP measurements that confirm a location having an annulus-typical mix of intracardiac EP signals blending atrial and ventricular patterns. Provided as displayed guidance, the identification may comprise, for example, color- or other image-based coding of locations on a structural image of the heart.

As part of SHD intervention device placement, a guided action optionally comprises an action for SHD intervention device actuation, deployment, and/or attachment. Attachment comprises, for example, applying force to tissue (for example, clamping over tissue and/or expanding to press against the walls of a lumen or aperture), and/or penetrating tissue.

Additionally or alternatively, guided actions verify and/or validate SHD intervention device placement and/or attachment. Optionally, for example, an SHD intervention device is itself used as an electrode to detect electrophysiological (EP) signals, and/or to pace EP activity detected from another electrode. These actions can provide information about what structures are near to the SHD intervention device. Optionally, placement of the implanted SHD intervention device is verified and/or validated using measurements from another probe; for example, contact of a mapping probe with the SHD intervention device potentially indicates a location and/or position of the SHD intervention device.

Optionally, a guided action comprises retrieval and/or at least partial de-implantation of an SHD intervention device; for example, an SHD intervention device which is incorrectly placed and/or non-functioning.

In some embodiments, integration of information into a common model is used to automatically identify conditions of particular concern and/or relevance to a guided action—for example, proximity to vulnerable structures, and/or verification based on a plurality of criteria that an SHD intervention device's placement is correct. The model is optionally presented as an image. Optionally, the guidance resulting from the integrated model is of a non-image type, for example, comprising text instructions, audible tones, and/or tactile indications such as shaking and/or increased resistance to movement of a user input device.

Coordinated knowledge of various functional components of the heart anatomy is used, in some embodiments, to guide the operator away from performing interventions in support of one heart functional component at a location or otherwise in a manner that might be detrimental to another heart functional component. Potential detrimental effects include:

Chronic (e.g. decubitus/pressure) or acute (e.g., trauma) injury to the AV node.

Transecting the bundle of HIS.

Disruption of the phrenic nerve.

Damaging the ganglionic plexi innervating the AV node.

Interrupting the right coronary artery (e.g., leading to severe ischemia and/or irreversible myocardial damage).

Interrupting coronary sinus flow (e.g., leading to severe impact on the mechanical performance of the myocardium).

Comprehensive information on the heart (e.g. mapping of the mechanical, electrical, hemodynamic and/or possibly other components) can suggest and/or guide interventions that are “multi-systemic” in their nature (e.g. changes in electrical activation patterns, together with hemodynamic valvular modification).

In some embodiments of the present disclosure, images and/or models which combine information about patient heart electrophysiology and detailed patient heart structure are presented to a viewer. In some embodiments, the information describes more than one heart functional component (e.g., valve movements and EP function together). Optionally, the information is updated in real time. In some embodiments, the information is updated to also show the position of an SHD intervention device and/or other device used in a MISHDI; optionally to show areas with functional properties which make them targets of treatment by the SHD intervention device, and/or to show areas with functional properties which make them important to avoid with the SHD intervention device. The image and/or model are optionally shown as a 3-D image of heart anatomy with functional property information (of one or more modalities) encoded with the anatomy.

An aspect of some embodiments of the present disclosure relates to the integration of multimodal measurements of an intrabody environment of a medical procedure for treatment of structural heart disease—including measurements of structure and of events taking place therein—into a compound model which unifies them (and, optionally, is used to produce a unified display). The compound model and/or a unified display produced therefrom is used, in some embodiments, to guide and/or monitor the procedure (which, in some embodiments, comprises a MISHDI).

Devices made use of which directly provide MISHDI treatment in some embodiments of the present disclosure, include, for example, one or more of the following:

LAAO (left atrial appendage occluder)

TAVI (transcatheter aortic valve implant)

Mitral or Tricuspid valve clip

Mitral or Tricuspid annuloplasty ring or band

ASD (atrial septal defect) closure device

It should be understood that this list does not exclude the use of other devices as tools during a MISHDI procedure, e.g., for tissue manipulation, navigation, and/or sensing. It should be understood that while overall descriptions are provided in terms of at least some of the just-mentioned specific MISHDI devices and/or their associated implantation and/or use procedures, there are also described general principles which relate to structural heart disease intervention more generally.

Herein, the term “multimodal measurement” refers to the use of measurements of a plurality of different types (e.g., measurement of two different types) to characterize the intrabody environment of the procedure and/or activities taking place within it. Any individual embodiment of the present disclosure optionally uses a particular set of a plurality of measurement approaches. Some descriptions of the present disclosure emphasize in particular the integration of measurements of EP signals, a category of measurements which itself spans a range of “measurement modes”, a terms which is further defined herein below.

Some embodiments of the present disclosure may be understood as establishing a “scaffolding” that structures position relationships among locations within a treated organ; and relative to which other information about the organ (for example, electrophysiological measurement information) may be localized. The scaffolding may distinguish just a few different locations (e.g., 10 or fewer, or locations along just one or two axes within a lumen of an organ). Optionally the scaffolding is a 3-D model of, e.g., organ lumenal surfaces. The scaffolding may be refined by the addition of measurements, optionally proceeding from an earlier, few-location state through to a later, 3-D surface or other detailed model.

The scaffold is optionally built based on earlier measurements in a procedure, or inputs from pre-procedure data sources. The scaffold at this stage optionally provides a basic model of a procedure's tissue environment—for example, extents of lumenal axes, and/or relative locations of major lumenal structures. Further measurements, in some embodiments, continuously provide further detail to and/or update that model, as they are associated to their appropriate locations within the compound model. The compound model develops over time as a result. The association of new measurements, in some embodiments, is performed in a manner that supports guiding and/or monitoring a procedure in real time.

The scaffolding of the basic model is optionally based on a primary measurement modality (for example, imaging by electrical field measurements, ultrasound intra-cardiac echocardiography, magnetic field imaging, X-ray, MRI, and/or CT), or built up by the coordinated use of a plurality of measurement methods. Measurements may be made using devices auxiliary to a device which directly provides MISHDI treatment (e.g., imaging probes placed within the heart lumen or at other locations). Optionally, a device which directly provides MISHDI treatment is itself used as a measurement device; for example:

conductive elements of the device such as control wires (e.g., an electrically insulated control wire with a selectively exposed conductive section) and/or fasteners are configured as electrodes, and/or

electrodes are attached to the device and/or positioned where they have a known spatial relationship to the device (e.g., on a sheath of a delivery catheter of the device).

Locations within the compound model are optionally described in terms of spatial coordinates (spatial positions) and/or distances; and/or in terms of non-spatial metrics which characterize a measurement, such as its signal phase, amplitude, and/or eigenvalue of one or more eigenvector components of the measurement (e.g., as determined by a method of mathematical decomposition). In some embodiments, a compound model includes both spatial and non-spatial representations. For example, EP measurements may be used to guide a procedure by the assessment of “similarity (of the measurement) to measurements expected at a target,” e.g., a target of the SHD intervention. “Spatial distance to a target” is optionally provided in coordination with the similarity assessment, e.g., to confirm and/or refine it.

Relating to MISHDI procedures in particular: embodiments of the present disclosure describe multimodal measurement-based solutions for problems which arise during the course of a MISHDI procedure, including problems associated with:

locating and/or identifying a region targeted for implantation of an SHD intervention device;

planning and/or actual implantation of the SHD intervention device which avoids damage to sensitive heart areas; and/or

planning, implementation and/or verification of attachment of the SHD intervention device to the heart.

A particular class of multimodal measurement-based compound models combines EP measurements of heart activity with detailed positional (including detailed structural shape) information. In some embodiments, intracardiac measurements of endogenous electrical activity are localized in space by coordinating them closely with locations (e.g., the “scaffolding”) defined by the compound model. Optionally, the locations are themselves further characterized according to their suitability as sites for SHD intervention device deployment and/or implantation; e.g., a determination that the device is at a location comprising fibrous tissue of the valve annulus during implantation of a TAVI, valve clip, or annuloplasty device. The electrophysiology reveals, in some embodiments, locations implantation should avoid (e.g., because certain electrically active tissue is particularly vulnerable to mechanical damage). This information is optionally used to exclude device attachment at sites which otherwise (e.g., mechanically) may appear to be suitable for attachment and/or implantation.

Compound models are optionally displayed as images; for example images which combine structural anatomy with functional anatomy such as EP measurement results, and optionally computer-processed interpretations of EP measurements: for example, the location of the AV node, the location of the bundle of His, the location of a heart valve structure such as the valve annulus, and/or position along an axis over which the EP measurements themselves vary, for example as a function of waveform component amplitude and/or timing.

Potential advantages of applying a multimodal measurement (and compound modeling) approach to a MISHDI procedure include reduction in how aggressive, risky, and/or expensive the overall procedure is. For example, in some embodiments, multimodal measurement provides information sufficient to guide the procedure without the use of methods that are performed with general anesthesia; for example, trans-esophageal ultrasound imaging. In turn, when general anesthesia is avoided, a requirement for artificial ventilation is potentially removed. Apart from adding to the complexity of the procedure, artificial ventilation has the effect of changing normal negative pressure breathing (sucking in air via movements of the chest and diaphragm) into positive pressure breathing (pushing air in artificially). Positive pressure, in turn, can have an effect on the shape of the heart, including shrinking heart chambers and/or valves that are normally more open and prone to regurgitation. Consequences of this can be that the heart structure is incorrectly adjusted and/or assessed intra-procedure. For example, an annuloplasty procedure performed during positive pressure ventilation potentially under-corrects; or if positive pressure effects are taken into account, may paradoxically over-correct.

In some embodiments of the present disclosure, multimodal measurement including structural measurements performed intra-procedure (for example using electrical field, magnetic, and/or ultrasound imaging), optionally augmented by EP measurements, removes a need to obtain a prior spatial map of the heart using CT or MRI imaging. Potentially, a need for planned open-heart surgery procedures and/or a risk of complications which lead to unplanned open-heart surge procedures is reduced.

An aspect of some embodiments of the present disclosure relates to the use of heart tissue electrical activity mapping to guide the placement of devices used to intervene in (treat) structural heart disease—for example, placement of implantable devices, and/or positioning of therapeutic devices used to remove and/or remodel tissue (optionally including induction of tissue removal/remodeling).

In some embodiments, the intervention comprises a treatment for a form of structural heart disease, for example, one or more of: mitral/tricuspid valve annuloplasty, mitral/Tricuspid valve clip placement, Left atrial appendage occlusion (LAA), transcatheter aortic valve implantation/replacement (AVI/R), occlusion of a patent foramen ovale (PFO), occlusion of an atrial septal defect (ASD), occlusion of an ventricular septal defect (VSD), and/or hypertrophic cardiomyopathy reduction. Herein, a minimally invasive treatment for structural heart disease in general is referred to as a minimally invasive structural heart disease intervention (MISHDI). Devices used in the intervention, in some embodiments, include implantable devices such as occluders, clips, artificial valves, annuloplasty rings, and/or devices which serve to deliver therapeutic agents, e.g. for ablation and/or stimulation.

For carrying out a MISHDI, in some embodiments, an operator is provided with imaging- and/or modeling-based visualizations which show them the position of one or more devices used in the MISHDI relative to anatomy of the heart, and in particular, anatomy at the site of treatment. For example, in the process of performing a MISHDI, an operator navigates an implant or other device to a target site within the heart, including assurance of a desired apposition of the device with the tissue at the target site.

In some embodiments of the present disclosure, a MISHDI carries with it a potential for interacting with heart tissue electrical function of the heart (e.g., electrical impulse generation, regulation, and/or transmission) as a side-effect of the MISHDI. Preferably, a MISHDI is carried out without inadvertent effects on heart tissue electrical function, while still producing selected effects on heart tissue function overall. However, the working area of the MISHDI potentially at least partially coincides with critical structural features of heart electrical function, leading to risk of complications such as:

Chronic (e.g. decubitus/pressure) or acute (e.g., trauma) injury to the AV node.

Transecting the bundle of His.

Disruption of the phrenic nerve.

Damaging the ganglia plexi innervating the AV node.

In some embodiments, a critical structural feature of heart electrical function is an anatomical structure which, if damaged, leads to debilitating effects on overall heart pacing, potentially including total block of heart pacing and/or change of heart pacing to a physiologically unsustainable rate.

In some embodiments of the present invention, anatomical mapping of the target site of the MISHDI is performed using a mapping electrical field measurement probe which is also configured to simultaneously (and/or in a rapidly multiplexing mode with switching of, e.g., about 100 Hz or more) make measurements of heart electrical function. In some embodiments, the measurements of heart electrical function are measurements of electrophysiological (EP) signals produced by the heart's electrical functioning. The measurements may be of EP trace data, and/or measurements of the timing, amplitude, or other characteristic of an electrical signal produced by the heart. The measurements relate, for example, to electrical impulse generation, regulation, and/or transmission.

In some embodiments, the mapping probe makes electrical field measurements of mapping electrical fields induced within a heart chamber by electrical field generating electrodes. These mapping measurements provide position information that is converted to an image and/or model of the anatomy of the target site. Positions associated with the heart electrical function measurements are then also located within this image and/or model, based on being measured at the same positions as the mapping electrical field measurements, e.g. simultaneously with them. From this information, heart anatomical structures associated with the heart electrical function are located within the same image and/or model, to produce a combined anatomical-electrical functional map.

When the MISHDI itself is carried out, position finding for one or more of the devices used to perform the MISHDI (optionally a device which is not originally itself an electrical field measurement probe) is optionally performed using the same type of mapping electrical field measurements. For example, measurements made by the MISHDI device (e.g., by an electrode placed on it, and/or by an electrically conductive portion of the SHD intervention device itself) are treated as though they were measurements made by the electrical field measurement probe (i.e., directly, or together with the use of a suitable transformation of the measurement and/or the map). In some embodiments, position measurements are made after the MISHDI device is at least partially deployed, using a probe which can also show and/or detect proximity to the MISHDI device. For example, the probe is mechanically linked to the MISHDI device. Optionally, the probe's measurements (e.g., of voltage and/or impedance) are characteristically affected by contact with, e.g., electrically conductive portions of the MISHDI device, so that it can be used to “find” the MISHDI device.

This allows position to be estimated—in particular, position relative to structures involved in heart electrical function—even if the SHD intervention device is not itself used to make heart electrical function measurements. Additionally or alternatively, analysis of heart electrical function measurements made using the SHD intervention device is constrained by previous measurements. This is optionally used, for example, to help determine a position of the SHD intervention device, and/or to help characterize the electrical function measurement as indicating a position near a critical element of heart electrical function.

Additionally or alternatively, in some embodiments, the SHD intervention device is located in the heart by another method; for example, X-ray, magnetic imaging, and/or ultrasound. The imaged position is located within the combined anatomical-electrical functional map, for example by registering anatomical features shown in the image with corresponding anatomical features in the combined anatomical-electrical functional map.

It is a potential advantage to make a combined anatomical-electrical functional map using measurements of different types (that is, mapping electrical field and heart electrical function) with a same probe, insofar as this reduces skew in heart electrical function localization due to the substantially simultaneous measurement of both position data and heart electrical function.

An aspect of some embodiments of the present invention relates to guiding placement of an implantable device and/or positioning of a therapeutic device (an SHD intervention device) to avoid damage to functional components critical to heart functioning, but not themselves planned to be targeted by the implantation/therapy. In some embodiments, the implantable and/or therapeutic device is a device for treating structural heart disease. In some embodiments of the present disclosure, measurements of heart electrical function are combined with position (that is, anatomical) measurements to help localize structures critical to heart electrical function, potentially assisting in avoiding harming them during implantation and/or placement and operation of the device.

An aspect of some embodiments of the present invention includes a method of indicating a positional relationship between an identified portion of heart tissue and a device configured to provide structural heart disease intervention therapy.

The heart tissue is, in some examples, a wall of a heart chamber, e.g., of a ventricle or atrium. In some examples, the heart tissue may be a heart tissue with specific role in the generation and conduction of electrical signals in the heart, for example, the AV node, the bundle of His, the right bundle, the left bundle, etc.

In some embodiments, the heart tissue is identified based on intracardiac electrocardiogram (IC-ECG) data measured by an electrode touching the tissue, or positioned in the vicinity thereof. The vicinity, in this context, is any place in which the electrical activity of the tissue may be measured by the electrode at a signal to noise ratio good enough to allow identification of the tissue based on the measurement. The IC-ECG may be unipolar or bipolar. In some embodiments, the bipolar IC-ECG signals are measured between two electrodes of an in-body catheter, for example, a lasso catheter, or any other electrode known from the field of electrophysiology. Preferably, the catheter carries a plurality of electrodes, to allow bipolar IC-ECG measurements, and optionally, to allow imaging as described, for example, in International Patent Publication No. WO2018/130974, International Patent Publication No. WO2019/034944, and/or International Patent Publication No. WO2019/035023; the contents of each of which are included herein by reference in their entirety.

In some embodiments, the tissue is identified based on a comparison between IC-ECG signals, and body-surface ECG (BS-ECG) signals. For example, various portions of the BS-ECG signal may be each related to a corresponding heart tissue, and an IC-ECG signal received simultaneously with a BS-ECG signal portion may be identified as being received from the tissue corresponding to the portion of the BS-ECG signal. This allows the BS-ECG signal to provide context to the IC-ECG signal (e.g., what other parts of the heart are activated before and after it), which acts to constrain where the IC-ECG signal could have been recorded from to a certain location (e.g., a position in space above, below, or between other electrophysiologically active tissues, and/or a location identified as comprising a certain tissue structure). Accordingly, in some embodiments, the heart tissue is identified based on comparison of ECG signals measured by the in-heart electrode during some time period, and surface ECG signals measured during the same time period. In this context, the BS-ECG signal comprises a reference signal, and the IC-ECG signal comprises an identifying signal.

In some embodiments, the reference signal is another IC-ECG signal. For example, a positional relationship is identified between an identifying electrode (e.g., the device used for the intervention, acting as an electrode) and some heart tissue location by accessing measurements (i.e., IC-ECG measurements) made by another in-heart electrode acting as a “reference” electrode. The reference electrode measurements are indicative of electrical activity of heart tissue in the reference electrode's vicinity. Where the electrodes' measurements are more similar, e.g., in amplitude and/or latency, the positions of the two are optionally estimated to be correspondingly closer together; where the two measurements are more different, the positions are optionally estimated to be correspondingly more separate. Optionally, relative latency allows identifying a location of the device used for the intervention along an axis of electrical activation “before” or “after” the position of the reference electrode.

In some embodiments, the method includes identifying a position of an electrode in space using a structural representation of the heart. The position may be, for example, in relation to an image of a heart or a portion thereof. In some embodiments, the image is of the heart of the patient, and may be taken in advance (for example, as a pre-acquired CT or MRI image) or during the intervention, for example, based on measurements of electrical fields generated by other electrodes, for example, as described in the above-mentioned International Patent Publication No. WO2018/130974, International Patent Publication No. WO2019/034944, and/or International Patent Publication No. WO2019/035023. In some embodiments, the position of the identified heart tissue is identified based on data indicative of the position of the in-heart electrode when the in-heart electrode measured the electrical activity of the heart tissue. For example, the in-heart electrode may be an electrode of a mapping catheter, used for generating the image as described in one of the above-mentioned patent applications, and its position in relation to the image generated is identified as described there. In other embodiments, the measurements may be registered with a pre-acquired image, for example, as described in International Patent Publication No. WO2018/078540, the contents of which are included herein by reference in their entirety.

The method of identifying the positional relationship between the device used for the intervention and the tissue and the identified heart tissue also includes accessing measurements indicative of a position of the device.

These measurements may be according to any method known in the art as such, including any tracking system associated with registration that fits measurements to positions in space. In some embodiments, the position of the device is made by using the device (or an electrode attached thereto) as an electrode for reading electrical fields generated in the heart chamber by other electrodes, and using these measurements for identifying the location of the device (or electrode), for example, as described in International Patent Publication No. WO2019/034944, and/or in U.S. Provisional Patent Application No. 62/898,581, the contents of which are included herein by reference in their entirety.

Once the heart tissue position and function are identified, and so is the position of the device, a method according to some embodiments of the present disclosure includes indicating, on a single image of a portion of the heart, the position identified for the heart tissue, the identification of the heart tissue, and the position of the device. The identification of the heart tissue may be indicated, in some embodiments, by coloring the image portion showing the heart tissue with a distinct color (or otherwise marking the image portion, e.g., with a texture, pattern, label, time-varying behavior such as flashing, or another indication making it distinct from its surroundings). The color (or other marking) may be allocated to the tissue in advance, so that the color obtains an identifying power for a physician used to working with a certain apparatus configured to carry out the invention. In some embodiments, there is a user interface, that allows a user to associate colors to tissues, thus facilitating adaptation the apparatus for different coloring conventions.

In some embodiments of the invention, the image of the heart, on which the positional relationship is to be marked, is partial, provided from a non-conventional view point, or hard to decipher because of any other reason. In such cases, it may be particular advantage to color (or otherwise mark) various features in the image to assist in orientation in the hard-to-decipher image, even if they don't have any particular importance for the intervention, for example, to color heart chamber walls that are away from the sight at which intervention is to take place.

In some embodiments, in the vicinity of the intervention site, there may be tissue that should not be intervened with, for example, because intervening with them may cause severe complications. Examples of such tissues may include, for example, the AV node and the bundle of His. Thus, in some embodiments, the heart tissue identified is tissue that should not be intervened with. This may decrease the risk of complications and reduce the cost of treating the patients.

As described elsewhere herein, the device configured to provide structural heart disease intervention therapy may include any one of an annuloplasty ring; a heart valve clip; a left atrial appendage occluder; a ventricular septal defect clip; a heart valve implant; and a heart valve replacement.

An aspect of some embodiments of the present disclosure relates to performing a MISHDI procedure using electrophysiological measurement-based guidance, the measurements for which also comprise an electrophysiological study.

In some embodiments of the present disclosure, EP signal measurements used as inputs to guide actions of a MISHDI are also EP signal measurements performed as part of an electrophysiological study.

Examples of electrophysiological studies include comprehensive EP evaluations which typically include a plurality of the following features:

Left and/or right atrial/ventricular pacing/recording

Recording (e.g., during pacing) from the bundle of His, coronary sinus, and/or left/right atrium/ventricle.

Intracardial use of multiple electrode catheters at different positions.

Induction/attempted induction of arrhythmia.

3-D EP mapping.

It is noted that certain combinations of these examples potentially correspond to present, historical and/or future “current procedural terminology” (CPT) code descriptions.

### Additional Definitions and Examples

A MISHDI procedure is performed, in some embodiments, using minimally invasive (e.g., over-catheter) techniques of delivery, positioning, deployment, and/or attachment. Approaches to the heart for minimally invasive approaches include, for example: vascular approaches via the inferior or superior vena cava; through arteries (e.g., from the carotid artery or by small chest incision); or in some embodiments through the apex of the left ventricle.

A range of problems (described further in the descriptions following) are associated with implantation of SHD intervention devices. These problems potentially interfere with safety, reliability, and/or effectiveness of the device and/or the procedure which implants it. In some embodiments, problems are potentially mitigated by the measurement and use of data which indicate aspects of the anatomical and functional environment of the device at the site of implantation, and/or aspects of the device itself.

With respect to the anatomical/functional environment, data may indicate, for example, overall heart lumen shape, overall heart function, heart structural anatomy, and/or heart functional anatomy. Although these categories are not dichotomous (the same data potentially belongs to more than one of these categories), the categories mentioned represent differences in emphasis.

In particular, data indicating heart structural anatomy optionally encompass one or both of (for example):

Through what regions and/or at what boundaries different regions of heart tissue are distinct in their mechanical and/or cellular-level properties. These optionally include, for example: the boundary between fibrous tissue of the valve annulus and surrounding cardiac muscle, the boundary between valve leaflets and the valve annulus, the course of transmission pathways such as the bundle of His, the positions of targeted transit points in heart septal walls, and/or positions of thrombic material.

Specifics of cardiac shape including the detailed shape and/or location of lumenal and/or perilumenal structures. These optionally include, for example: papillary muscles, chordae, valve leaflets, valve annuli, cardiac structures specialized for impulse transmission (e.g., sinoatrial (SA) node, atrioventricular (AV) node, and/or bundle of His), blood vessels supplying and draining the cardiac tissue (coronary arteries, coronary veins), other major blood vessels, septal wall geometry including fossae and/or the foramen ovalis, and/or atrial appendage geometry.

While some of these may be deduced in part from overall heart lumen shape, “overall heart lumen shape” as such refers herein to the (optionally time-varying) shape of the lumenal wall boundary as such (e.g., the outer bounds within which movement of an object within the lumen is confined), without specific reference to tissue properties.

Data indicating heart functional anatomy optionally encompass one or more of (for example):

Passive and/or active details of how structures move; for example, movements of valve leaflets and/or contractions of cardiac muscle.

Electrical activity of cardiac tissue (typically measured from EP signals), optionally including variations over time and space.

Measurements that provide a metric for a function attributable to a specific heart structure: for example, a measurement of the backward flow of blood through the defective tricuspid valve (known as tricuspid valve regurgitation) may characterize the functional anatomy of the tricuspid valve, not necessarily with associated structural detail. The tricuspid valve regurgitation may be measured, for example, by sensing backflow of an injected tracer fluid such as saline or dye.

Metrics and/or categories of anatomy and/or movement associated with specific structures, and carrying special meaning for the operation of the heart and/or for planning of a MISHDI procedure. For example, an open area of the tricuspid valve when it is maximally closed is a structural characteristic of the valve, but optionally also a metric of regurgitation. In another example: a percentage of shortening of the papillary muscles during the cardiac cycle (summarizing their dynamic motion) has potential implications for valve function such as risk of prolapse. In a further example: the left atrial appendage assumes different shapes in different hearts, optionally categorized as, for example, “windsock”, “ chicken wing”, “cauliflower” or “cactus”. Choice of approach and/or device used to close off an LAA may differ, e.g., depending on the shape of the LAA which should receive an LAAO device.

In contrast to such structurally-associated data, data characterizing “overall heart function” includes, for example: aspects of body surface (BS-) ECG recordings (although details of BS-ECG recordings may also reveal functional aspects of specific heart structures), heart rate, and/or overall pumping volume of the heart.

Herein, data addressing any of these indications of structural anatomy, functional anatomy and/or overall heart function, together with an analysis procedure used to extract specific information from the data, are considered to be comprised in a single “measurement modality”. Measurement modalities are not necessarily fully segregated from each other. For example, in some embodiments, the same tool (e.g., electrode-carrying probe), and optionally even the same stream of raw measurements (e.g., a stream of voltage measurements from an electrode of the electrode probe) is used as the basis of a plurality of measurement modalities. In such embodiments, the measurement modalities are distinguished from each other, for example, by different algorithmic processing, by auxiliary information used, and/or by how outputs are integrated to the multimodal model and/or presented for display. It should be understood, however, that measurement modalities are optionally grouped together according to their commonalities; for example, the measurement device and/or source of signal energy being measured (for example: electrodes/electrical fields, magnets/magnetic fields, ultrasound transducer/ultrasound generator, X-ray sensor/X-ray source).

With respect to an SHD intervention device itself, data may indicate, for example: device position, device deployment status, and/or device attachment status. Device position data optionally indicate offset in space along one or more axes and/or orientation in space in relation to one or more axes. Optionally device position data indicate distance and/or angle from a reference position. The reference position can be, for example, a transmitter, a distinguishable anatomical location, and/or a location distinguishable on the basis of a measurement that can be made while occupying and/or in close proximity to it.

Data may also indicate, directly or indirectly, how the device interacts with the anatomical environment, e.g., to affect (actually, as estimated, and/or as predicted) cardiac function. The interactions measured are potentially intended and/or unintended; therapeutic and/or adverse.

In some embodiments of the present disclosure, implantation and/or validation of implantation is guided and/or monitored using within-body imaging and/or sensing devices and methods. In some embodiments, this is without the use of ionizing radiation (e.g., without the use of X-ray and/or radionuclide-based imaging). Additionally or alternatively, in some embodiments, this is without the use of imaging data acquired pre-procedure; for example, all the data used for modeling and/or visualization is acquired as part of the MISHDI procedure itself.

A particular potential advantage provided by some embodiments of the present disclosure is conferred by use of electrode measurements to provide a plurality, and optionally substantially all, of the data used in the different measurement modalities. For example, an electrode used in mapping positions within a cardiac lumen is optionally used also for sensing:

differences in dielectric and/or impedance properties characteristic of different tissue structures, and/or contact therewith,

dielectric signals characteristic of an injected tracer (such as saline),

intrinsic cardiac electrical activity (EP function), and/or

electrical signals transmitted by a marker device such as a wire inserted to the coronary artery.

Potential advantages of using electrical measurements from electrodes (e.g., on probes and/or SHD intervention devices), compared to other intrabody probe types, include: reduced numbers of probes, and/or increased simplicity of probes. Optionally, a catheter conveying the SHD intervention device, the SHD intervention device itself and/or attachment hardware of the SHD intervention device include at least one of the measurement electrodes; potentially reducing a number of catheters to be inserted during the procedure. There is also a potential advantage for data integration to the compound model: for example, two different measurement modalities can be directly identified as characterizing to the same location, insofar as it was recorded by the same probe at the same time. There are also potential advantages in terms of implementation, by reducing complexity in coordinating between disparate measurement devices.

Some descriptions herein relate to a respective operation (action/s within a procedure) performed in a certain manner, to achieve some particular intermediate result of an overall implantation procedure. It is to be understood that such operations are optionally performed within any suitable overall procedure, and performed, moreover, in any suitable combination with other operations of the procedure, and in any suitable order, as may be selected to achieve the implantation. For example, some operations are described as one option among a plurality of options for accomplishing the same intermediate result; any procedure which includes that intermediate result optionally uses any of the plurality of options. In some embodiments, the intermediate result itself is an optional part of the overall procedure—for example, an operation to verify position and/or attachment may be performed optionally. Moreover, in some embodiments of the present disclosure, all or portions of operations optionally occur sequentially (and the particular order of the sequence itself is optionally defined) and/or in parallel (e.g., simultaneously) with each other.

Herein, the term “electrophysiology signal” (EP signal) refers generally to any signal indicating heart electrophysiological activity. An EP signal is optionally recorded using measurements from one or more intracardiac electrodes (e.g., intracardiac electrograms) and/or using body surface electrodes to make ECG measurements. EP signals may be received raw and/or pre-processed, for example, pre-processed to identify (and/or otherwise annotate) signal regions of conventional significance (e.g., QRS complex), and/or of significance specific to interpretive use within a MISHDI procedure.

Examples of intracardiac measurement electrode configurations include unipolar (one electrode), bipolar (two electrodes), and vector (three or more electrodes) measurement configurations. It should be understood that any given electrode configuration comprising a plurality of electrodes is optionally used in any subset configuration of one or more fewer electrodes; conversely, electrodes used in a single electrode configuration are optionally used also in one or more electrode configurations including a plurality of electrodes. In some embodiments, intracardiac measurements are provided from existing implanted devices, for example, pacemaker and/or defibrillator leads.

Adding more electrodes to a measurement configuration allows making differential measurements between the two electrodes. This provides potential advantages for determining relative timing and/or relative amplitude of electrophysiological signals reaching each of the electrodes. In turn, this information may be used to estimate such characteristics as direction of signal propagation and/or presence of block between two electrode locations. Differential measurements also provide a potential advantage for cancellation of common noise, and/or for isolating EP signal features arising specifically from local electrophysiological activity.

Types of measurements made for any of these configurations optionally include one or more of the morphology (waveform “shape”, optionally including changes to the waveform shape), amplitude, slope (e.g., upstroke slope), duration, and/or spectral content of the measured electrophysiological signal. Any of these type of measurements (also referred to herein as metrics) may optionally be accessed by a processor. Optionally any of these types of measurements may be processed by a processor to identify changes over time, e.g., changes resulting from a structural heart disease intervention, the changes being measured at a plurality of different times. Signal amplitudes may be calculated, for example, as peak-to-valley distances and/or RMS amplitude. Slope may be calculated, for example, as the ratio of elapsed time and change in amplitude along a selected sloping region of a measured signal. Changes in waveform shape may be calculated, for example, using any of the foregoing methods, optionally in combination, or by another method such as peak counting, peak width, and/or peak area calculation. Duration may be calculated between defined landmarks of a signal component's waveform; for example, between measurement times differing by at least 10% from a baseline reference. Spectral content may be measured, for example, by use of Fourier analysis or another signal decomposition method. Changes may be evaluated on calculated metrics by processing such as subtraction and/or ratio calculation for a plurality of measured instances of the metric.

Unipolar measurement configurations in particular are optionally used for injury current measurements, for example due to flow of current from a damaged area of a heart to a normal area of the heart between heart beats. The use of a unipolar configuration has a potential advantage for such measurements, since the flow of current may be occurring similarly throughout the heart, such that a differential measurement would potentially mask the injury current's amplitude by being too similar at each electrode for clear detection.

Body surface ECG signals are optionally recorded in different electrode and/or virtual electrode configurations, for example 12-lead ECG (comprising, for example, ten physical electrodes; the other leads are typically generated as special combinations of measurements from the physical electrodes) and 3-lead ECG (comprising, in some embodiments X, Y, and Z axis components calculated from combinations of measurements from electrodes optionally placed as for 12 lead ECG). Optionally, body surface ECG signals are measured and/or analyzed along with intracardiac measurements to measure EP signals. The 3-lead ECG configuration is typically used for the production of vector cardiograms.

Aspects and/or components of a body surface ECG signal or other EP signal used, in some embodiments of the present disclosure, include rate, rhythm (e.g. distinguishing normal, ventricular premature beat, atrial premature beat, ventricular tachycardia, and/or heart block), heart rhythm variability, QRS duration, P wave morphology, PR interval, ST segment (duration and/or elevation), T wave morphology, and/or QRS spectral content.

Further aspects of EP signals measured, in some embodiments, include one or more of the following. In some embodiments, conduction time is measured between electrodes, locations, EP signal components, and/or between the delivery of a pacing signal and the arrival of activity induced by the pacing signal at one or more locations. Conduction speed is determined, e.g., as the ratio of distance between measurement locations and the conduction time. Patterns of conduction may be determined, for example, from conduction times, speeds, and/or the presence of conduction block; optionally including, for example: 1^{st} degree, 2^{nd} or 3^{rd} degree AV (atrioventricular) block. Examples of more particularly identified blocks include right/left bundle branch block (RBBB, LBBB), and left anterior/posterior fascicular block (LAFB/LPFB), also referred to as left anterior/posterior bundle of His block (LABHB/LPBHB). Timing (whether or not it comprises timing of conduction) optionally is measured between two EP signals, and/or between an EP signal and some action performed as part of a structural heart disease intervention (SHD intervention), for example, an action of an SHD intervention as described herein.

Any of these EP signals, EP signal aspects, and/or EP signal components is optionally displayed as-is; e.g., as a graphed trace and/or number(s). Optionally, they are displayed in real time (e.g., as they are acquired). In some embodiments, they are displayed or re-displayed after the fact (retrospectively), e.g., according to the position of an SHD intervention device or probe that is at or approaching the same location (e.g., as known from non-EP measurements) as the EP signal itself was first acquired from. Optionally “real time” and “retrospective” signals are shown together to allow changes to be seen, and/or a comparative analysis of their properties, is shown; e.g., a heartbeat phase-aligned difference.

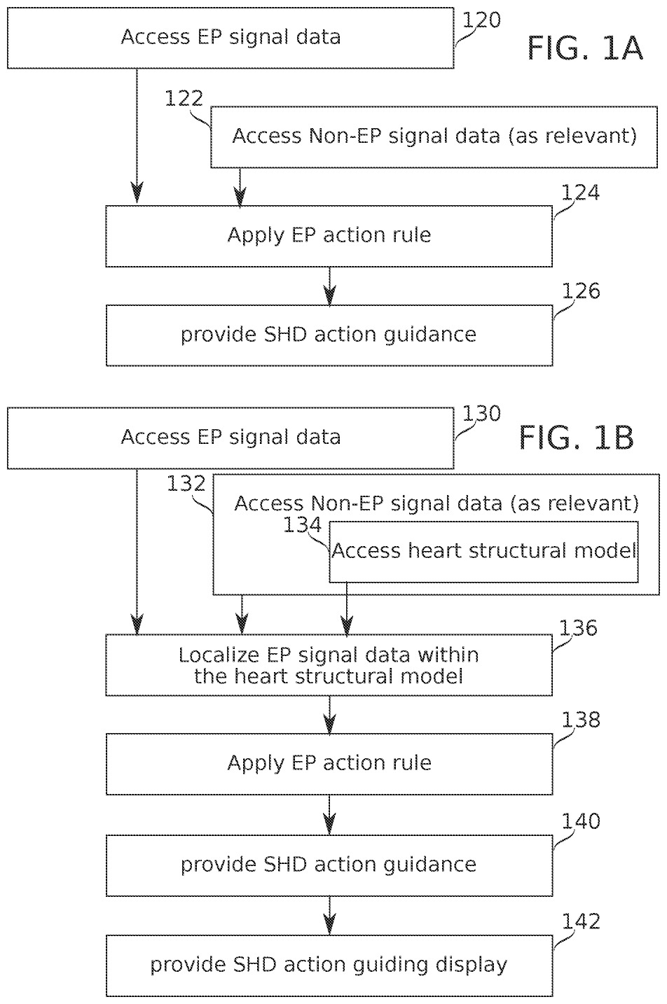

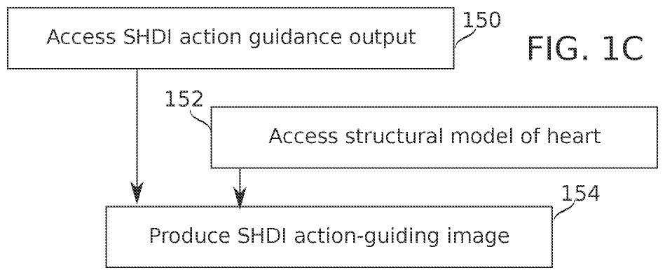

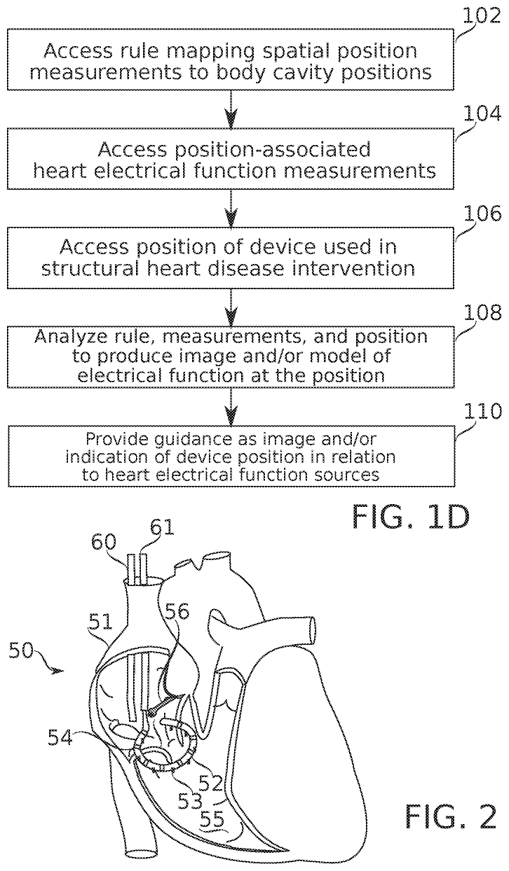

Additionally or alternatively, they are used as input to an EP action rule (defined, for example, in relation to FIG. 1A), which outputs guidance determined on the bases of EP signal inputs and optionally other non-EP inputs. In some embodiments, the use of a particular EP action rule is gated to the stage and/or state of the procedure, for example as described in relation to FIG. 1A.

Generally, characteristics and/or signal features determined from analysis of intracardially (and locally) recorded EP signals and/or body-surface recorded EP signals, optionally include one or more of:

Determinations of local activation time, e.g., relative to a BS-ECG, another intracardially recorded EP signal, and/or relative to a local activation time recorded previously, e.g., before an SHD intervention procedure, and/or before some particular action within an SHD intervention procedure.

Determinations of a change in an intracardiac electrogram more generally; for example, compared to another local electrogram, and/or compared to its previously recorded value.

Determinations of a change in a BS-ECG; for example, compared to another BS-ECG lead, and/or compared to its own previously recorded value.

Determinations of a change in an electrogram associated with a particular heart electrical system structure; for example, compared to another heart electrical system structure, and/or compared to its own previously recorded value.

Determinations of the result of a pacing test; for example a change of an EP signal and/or another measurement's value with and without pacing.

Any of these determinations is optionally displayed as-is, e.g., as a graphed trace, and/or number(s).

Additionally or alternatively, they are used as input to an EP action rule (defined, for example, in relation to FIG. 1A), which outputs guidance determined on the bases of EP signal inputs and optionally other non-EP inputs. In some embodiments, the use of a particular EP action rule is gated to the stage and/or state of the procedure, for example as described in relation to FIG. 1A.

In some embodiments, EP signals are analyzed to isolate characteristics and/or signal features due to particular structures of the heart's electrical conduction system. Examples of such structures include: the SA node, the AV node, the bundle of His, the crista terminalis, the bundle of Bachman, the right bundle, the left bundle, and/or the Purkinje fiber(s). Isolation is optionally performed, for example, by comparison to a reference signal (e.g., a signal that is known in the art to be characteristic of a particular structure). Optionally, comparison is made between signals originating from two or more structures, and relative timing of activation is used as part of feature isolation (e.g., more ventricular structures activate after more atrial structures in a normal heartbeat pattern). Optionally pacing (e.g., from the position of a known structure) is used to determine an activation offset, which is potentially characteristic of another particular structure. Output from such an isolation, in some embodiments, comprises identification that the EP signal was recorded from a position at or near the particular structure of the heart's electrical conduction system. This output is optionally used together with non-EP measurement data (e.g., heart structure and/or intracardiac electrode localization information) to furthermore identify that a certain location, e.g., in 3-D space, is at or nearby a structure of the heart's electrical conduction system. Optionally, the isolated EP signal is furthermore characterized by value. For example, the stronger the structure-specific EP signal is (e.g., relative to some reference value and/or a currently available “maximum signal”), the closer the recording position is considered to be to the structure. Additionally or alternatively, a decrease in the strength of the signal feature (e.g., as re-measured at a same location) potentially indicates damage or other interference with electrophysiological function.

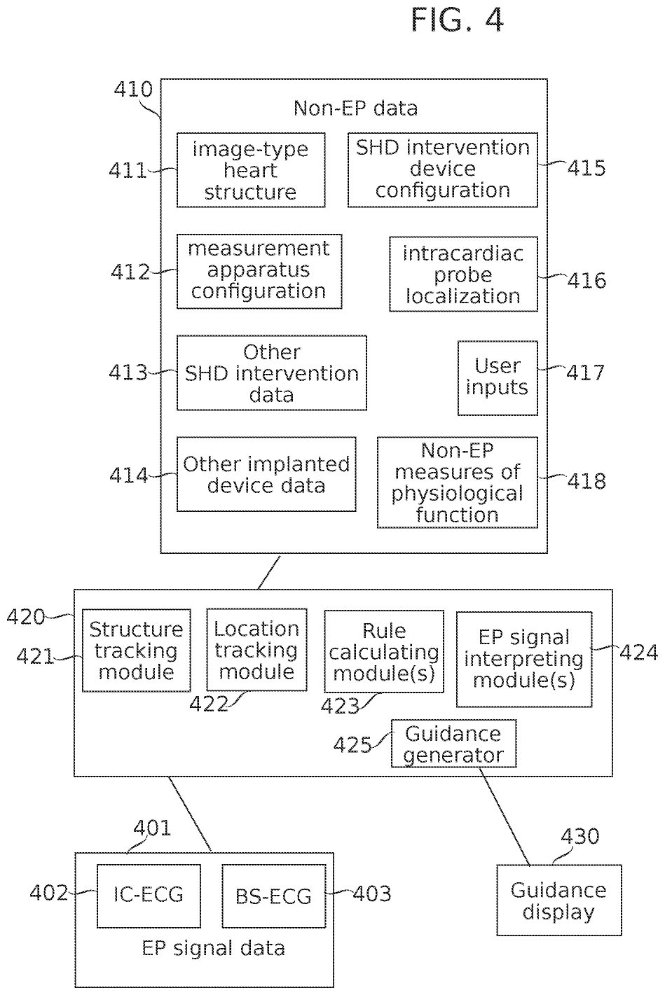

Herein, the term “non-electrophysiology” (non-EP) measurement, data, and/or input refers generally to members of a wide class of information used, in some embodiments of the present disclosure, for roles in sub-classes including sub-classes described in relation to sub-blocks 411-418 of block 410 of FIG. 4, herein.

Abbreviations

Herein, the following abbreviations are used:
EP electrophysiological
SHD structural heart disease
MISHDI minimally invasive structural heart disease intervention
ICE intra-cardiac echocardiography
MRI magnetic resonance imaging
CT computed tomography, specifically X-ray computed tomography
AV atrioventricular
SA sinoatrial
LAA left atrial appendage
LAAO left atrial appendage occluder
TAVI transcatheter aortic valve implant
PFO patent foramen ovale
VSD ventricular septal defect
ASD atrial septal defect
ECG electrocardiogram
BS-ECG body surface electrocardiogram
IC-ECG intracardiac electrocardiogram

Before explaining at least one embodiment of the present disclosure in detail, it is to be understood that the present disclosure is not necessarily limited in its application to the details of construction and the arrangement of the components and/or methods set forth in the following description and/or illustrated in the drawings. Features described in the current disclosure, including features of the invention, are capable of other embodiments or of being practiced or carried out in various ways.

Reference is now made to FIG. 1A, which is a flowchart schematically representing a method of producing computer-generated guidance for a structural heart disease treatment (e.g., treatment via MISHDI), according to some embodiments of the present disclosure.

At block 120, in some embodiments, a processor accesses EP signal data measured from one or more EP signals. Examples of EP signals and EP signal measurement configurations are given herein, for example together with the definition of “electrophysiology signal”, including IC-ECG and/or BS-ECG EP signals, measurements of such signals, and signal measurement configurations.

At block 122, in some embodiments, a processor optionally accesses one or more non-EP measurements. The non-EP measurements are optionally selected from among any one or more of the classes including, for example: image-type heart structure information; intracardiac electrode localization information; measurement apparatus and/or SHD intervention device configuration information; SHD intervention device status, procedure tool status and/or procedure status data; user inputs; non-EP measures of physiological function; and/or information from already-implanted devices. Examples of these classes are described, for example, together with the listing of classes provided hereinabove. Instances of non-EP measurements are also described herein in relation to particular embodiments of methods according to the present disclosure.

At block 124, in some embodiments, the processor applies an EP action rule to the one or more measured EP signals and optionally the non-EP measurements. Herein, an “EP action rule” comprises a rule defined for a processor; for example, defined as a program and/or data for the program, such as in a table and/or as weights of a machine learning result. The rule operates so that the processor transforms inputs of blocks 120 and optionally block 122 into an output (block 126) which has particular significance as guidance to an action of MISHDI procedure. More particularly, in some embodiments, the guidance applies to a current and/or next action of a MISHDI procedure, in view of a current status of the MISHDI procedure.

EP action rules apply to produce action guidance for MISHDI actions optionally including using EP signals (and optionally non-EP signals) to, for example:

Find and/or validate position at a particular heart structure for purposes of targeting it (e.g., for attachment and/or deployment of an SHD intervention device).

Find and/or identify a particular heart structure for purposes of avoiding it (e.g., to avoid damaging it with SHD intervention device).

Evaluate an EP signal change (that is, a change in a parameter of the EP signal) for signs that a previous action has caused, is causing, or is at risk of causing damage, potentially leading to a new action to reverse and/or mitigate effects of the previous action. Examples of such changes include appearance of conduction delay and/or conduction block. Other examples include changes to P-wave morphology, change in local activation time of a reference location of the heart, a change in the vector electrogram, and/or a change from a typical cardiac EP system component's electrogram. One mechanism potentially underlying such damage or potential damage comprises mechanical compression by an SHD intervention device affecting EP function.

Evaluate an EP signal change (that is, a change in a parameter of the EP signal) for signs that a previous action has resulted in an intended or expected effect associated with success of the action, potentially leading to a new action that does generate the intended/expected effect (if it is not yet achieved), or to proceeding to the next stage of the procedure (if it is achieved). Examples of such changes include at least partial block of electrical signal to the LAA after successful deployment of a LAAO device, for example as measured by changes in conduction time, P wave morphology, and/or local activation time. In another example, closure of septal defects potentially alters conduction times. One mechanism potentially underlying such effects comprises isolation of a tissue compartment from electrical activation by mechanical compression. Another comprises electrical modification of signals received in a tissue compartment by interposing a structure which modifies (e.g., increases or decreases) the normal transmission of electric currents.

The above four types of action guidance may be summarized as: identifying locations for SHD intervention device (1) targeting or (2) avoidance, and validating (evaluating) results of SHD intervention device deployment with respect to (3) intended and/or (4) unintended results. The guidance provided by an EP action rule is optionally of a fifth neutral type in terms of targeting/avoidance (e.g., a structure is identified as an optional waypoint and/or other type of orienting indication). In some embodiments, the evaluating of an EP signal change comprises comparing an EP signal parameter value associated with a baseline condition (that is, pre-action, for example before an SHD intervention device deployment) to a measurement of the EP signal parameter value measured during a later condition. The baseline condition EP signal parameter value is optionally a previously measured value of the same heart. Optionally, the baseline condition EP signal parameter value is a standard value, e.g., a value which is expected and/or typical of the EP signal parameter as known from the particular patient's prior history, and/or known for a population considered representative of the particular patient based on, e.g., age, sex, disease stage, and/or other parameters correlated with the EP signal parameter.

Targeting/avoiding optionally comprises indications which are spatial (e.g., represented in terms of distance and direction) and/or parametric (e.g., represented in terms of where a certain measured value falls within a range of relevant values). Spatial indications, in some embodiments, use a heart structural model, for example as further described in relation to FIG. 1B, and/or block 421 of FIG. 4. Parametric indications are optionally category-based. For example the position of an SHD intervention device may be indicated as “close” or “not close” to a potentially damage-vulnerable structure of the heart's electrical system based on passing a threshold of similarity of SHD intervention device-measured signals to a model signal. In another example, position along an atrioventricular axis is optionally categorized as “atrial”, “ventricular”, or “valvular”; optionally, the categorization is based on a threshold of similarity. In some embodiments, categorization is performed jointly together with other inputs, for example, image-based and EP signal-based measurements. Additionally or alternatively, parametric indications are quantitative; for example, based on amplitudes, differences of component amplitudes (e.g., P-wave amplitude vs QRS-wave amplitude), or another measure.

Effects indicated by EP signal measurements are optionally non-spatial. For example, increased latency and/or decreased amplitude of activity measured at a fixed location from a component of the heart's electrical conduction system is optionally treated as an indication of block whatever the spatial situation. Optionally, however, spatial information is in interpreting the EP signal—for example, a moving measurement electrode may measure different latencies and/or amplitudes depending on its position, and knowing its spatial location optionally assists in taking this into account.

The types of action guidance optionally apply to any MISHDI procedure, for example, LAAO, TAVI, valve clip and/or annuloplasty device implantation. Optionally action guidance is available for one or more actions of the MISHDI procedure, for example, according to a stage and/or state of the procedure.

EP action rules are optionally chained together; for example, in some embodiments, EP action rules guide the performance of one or more types of supporting action that provides input to another EP action rule. For example, a pacing action is to be performed to elicit EP signals that are measured from an SHD intervention device, to help determine if an SHD intervention device is beginning to induce block as it deploys, for example, because it is placing pressure on a heart electrical system structure as it deploys. Pacing is suitable for this, for example, because triggering timings can be precisely known, allowing changes in propagation time to be clearly measured. The pacing action itself is optionally initiated from an electrode positioned at a place in the upstream of the SHD intervention device. An EP action rule used in such a case could be a targeting-type rule (for positioning the pacing electrode, e.g., at the SA node), in support of the pacing measurement which is provided as input to an EP action rule for converting the EP signal that reflects pacing responses into guidance indicating a potential unintended result of SHD intervention device deployment.

From another perspective, EP action rules are optionally grouped in terms of particular types of EP input that they receive. General examples include measurements from and/or using implant parts (during pre-implantation movements and/or after implantation), evaluation of BS-ECG changes and/or events, evaluation of IC-ECG changes and/or events (e.g., indicative of damage), and evaluation of IC-ECG component signal source proximities and/or signal timings (e.g., indicative of locations of the sources and/or of the measurement device itself). Herein, reference to a signal source in relation to measurements of heart electrical function (e.g, measurements of EP signals) refers to a tissue location at which local activity (e.g., electrical activity) is what gives rise to emission of the signal being measured and/or a relevant component thereof. The source of a signal component is optionally identified as the region at which the amplitude of the signal component is strongest. In the case of a differential measurement, there may be a plurality of “signal sources” involved. Optionally, one is considered a baseline, background, and/or reference signal; and the other is treated as the signal source. Optionally both are considered signal sources. Examples of signal sources include electrically active bundles and/or nodes of the heart's electrical transmission system, for example, the bundle of His, the Purkinje fibers (including selection sections thereof), the atrioventricular node, and the sinoatrial node. Optionally, neural ganglia and nerves are signal sources. Optionally, portions of muscle itself (e.g., myocardial tissue) are signal sources. In a measured signal with many different signal sources, signal components may be identified by features such as amplitude, polarity, timing, and/or frequency content. In some embodiments, signal components are identified by knowing some aspect of the measurement conditions, e.g., the placement of a measurement electrode.

It should be understood that described indications can be optionally recruited to use in any suitable type of action guidance; e.g., the different methods of obtaining an indication of structure proximity may be for avoidance (and/or confirmation of sufficient distance), targeting (and/or warning of insufficient proximity), or neutral, depending on circumstances.

More particularly, in some embodiments, optionally one or more of the following types of EP measurements is used as input to an EP action rule:

EP measurements from an SHD intervention device are optionally unipolar or bipolar electrogram signals from the implant or a portion thereof; for example a tip of the implant, a fastener used with the implant (e.g., a screw), and/or an electrode fastened to the implant. In some embodiments, a sheath or other portion of the delivery apparatus of an SHD intervention device is provided with an electrode used for electrogram signal measurement. EP signals may be natively triggered or paced. Examples of indications extracted by an EP action rule and provided for use in action guidance and based on such EP measurements include, for example:

An EP signal amplitude change optionally provides an indication of tissue contact or loss of contact (e.g, a rise upon contact, or a fall upon loss of contact).

EP signals characteristic of heart electrical system components provide indications of proximity to these components (e.g., bundle of His, left bundle), and optionally indications of changes in proximity as the strength of the characteristics signals changes.

Additionally or alternatively, EP signals characteristic of heart electrical system components provide indications that they are continuing to function normally—or are potentially being subjected to damage such as may induce block and/or transmission delays.

Conversely, an SHD intervention device is used as a pacing electrode. This may include an electrically conductive portion of an SHD intervention device, SHD intervention device fastener, and/or an electrode associated to the SHD intervention device. Additionally or alternatively, the SHD intervention device's sheath is used as a pacing electrode. Examples of information provided for use in action guidance from pacing include:

Ability to pace from implant provides an indication that implant is close to the conduction system.

A change in BS-ECG and/or activation sequence in left atrial electrograms in response to pacing provides an indication that the implant is affecting the electrical system (potentially to its detriment, e.g., if slowing or otherwise becoming disordered, and potentially to its benefit, e.g., if improvement in general heart function as a result of SHD intervention indirectly improves EP function). In LAAO implantation procedures, the change in response to pacing may indicate that the LAAO is acting as a partial block on pacing input propagation, which is potentially an indication of increasing isolation of the LAA from the rest of the heart. Changes in threshold without other actions ongoing may indicate unstable placement of a device.

A change in the threshold of pacing provides an indication that heart EP function is being impaired (e.g., if threshold is increasing), or assisted (e.g., if threshold is decreasing). In an LAAO implantation procedure, an increasing threshold may indicate that the LAAO is acting to isolate the LAA from the rest of the left atrium. Changes in threshold without other actions ongoing may also indicate unstable placement of a device.

Examples of information provided from a BS-ECG for use in action guidance include:

Changes in characteristics of characteristic waves and/or intervals between waves. For example, AV transmission delay may be an unintended effect of treatments such as TAVI and/or annuloplasty; indicated, for example, by a change in QRS wave morphology, duration and/or spectra; PR interval lengthening; and/or conduction delay to the RV electrode. Implantation of an LAAO device potentially affects EP characteristics of the left atrium, indicated, for example, by a change in P wave morphology and/or spectrum.

Changes in response to pacing potentially indicate partial block (e.g., as an unintended result of TAVI); for example: changes in the difference of conduction time to atrial activation vs. conduction time to ventricular activation. Pacing is, for example, from the sinus, atrial pacing, and/or ventricular pacing.

Changes from baseline rhythm/arrhythmias a potentially indications of an issue such as a valve clip banging on the ventricular wall (and interfering with the previous heart rhythm), particularly with respect to the implantation of a mitral valve clip. Such changes include, for example, a new premature ventricular contraction, a short run of ventricular tachycardia or full-blown ventricular tachycardia.

Examples of information provided from IC-ECG (that is, IC-ECG measurements not using the SHD intervention device itself) include:

Changes in characteristic intracardiac EP signals from heart electrical system structures such as the bundle of His, left bundle, and/or Purkinje potentially indicate block and/or damage occurring along transmission pathways.

Increasing delays in intracardiac EP signals potentially indicate block and/or damage. For example: the coronary sinus electrogram may show lengthening of AV time interval (e.g., in association with TAVI and/or annuloplasty device implantation), and/or the latency to activation of the left ventricle may increase in association with a TAVI procedure.

In other instances, increasing relative delay and/or decrease in EP signal components measured between monitored sites potentially indicate effective implantation; e.g., of an LAAO, comparing sites within the LAA to sites outside the LAA (e.g., the pulmonary artery). Stabilization of a new delay may be an indication of stable implantation.

In some embodiments, intracardiac EP mapping is used, optionally including activation time-based classification of structures such as the atrium, ventricle, bundle of His, left bundle, Purkinje fibers, and/or AV ring. Additionally or alternatively, mapping is performed using pacing of, e.g., the bundle of His and/or AV node. This can distinguish, for example, fast and slow pathways of the AV node, according to the timing of conduction pathway potentials.

Optionally, the EP action rule is itself accessed and/or selected for application as an intermediate result of a computer program and/or machine learning result which receives inputs indicating a state of a procedure. The section is based on, for example: user inputs, SHD intervention device location, and/or SHD intervention device status. More than one EP action rule may be active during a given period of the procedure.

Optionally, the selection of the EP action rule to be applied is performed using a “state map” of a procedure, which brings into play different EP action rules according to different states. The states optionally correspond to different stages of a MISHDI procedure. There may be more than one state available for each stage (e.g., to cover different eventualities of the procedure). Optionally a plurality of states are active simultaneously, e.g., to cover conditions in different parts of the heart, or conditions of different tools, and/or different parts of an SHD intervention device. Optionally the state map is at least partially organized according to a linear or branched “state tree”, which orders procedure states according to earlier and later stages of the procedure, and optionally provides for branching into different parts of the state tree according to events and conditions encountered during the procedure.

It is a potential advantage for automation of guidance to divide a procedure into stages and/or states: for example, this helps to focus displays presented to a user on currently relevant information, it helps to modularize programming tasks, e.g., allowing irrelevant EP action rules to be disregarded as an inherent feature of program structure.

States and/or stages of a procedure are proceeded through, in some embodiments, based on triggers which may comprise user input confirmation, user input selection, and/or automatic detection of a state of the SHD intervention device or another tool of the procedure. Optionally, automatic detection comprises, for example, automatic detection of a sensed deployment, release, contact, tissue insertion, and/or wall crossing of an SHD intervention device or tool; detection of a particular feature or feature combination of an EP signal; and/or detection of a feature or feature combination of a non-EP measurement.

At block 126, in some embodiments, the processor provides action guidance output from the EP action rule applied at block 124. The action guidance as such optionally comprises internal state of a program and/or data structure, for example, a value of flag register, value of a variable, program event, intra- or inter-program message. In some embodiments, action guidance is part of and/or converted to an action guidance display, which comprises something presented to the senses of an operator such as an image, text, and/or an auditory and/or tactile cue. Examples of such conversion are further detailed, for example, in relation to FIG. 1C.

Examples of embodiments comprising SHD intervention device usage guided based on EP measurements as part of a MISHDI, include, for example, one or more of the following. Block numbers of FIG. 1A are given to show how each example may be understood as carrying out the method of FIG. 1A. Applicability of FIG. 1A is not limited to the stated conditions of each MISHDI procedure; rather, the examples are of the equivalent of a single “state” or “stage” of the procedure, for example as explained in relation to block 124.

LAAO (left atrial appendage occluder): Potentials measured from within left atrium and/or left atrial appendage are accessed (block 120), and processed (block 124) to confirm (block 126) that the potentials are consistent with a location within the correct target (the LAA). The result is optionally presented as a display indication that the doctor is implanting the occluder inside the appendage, rather than another location which somehow anatomically “mimicked” (e.g., in the accessed non-EP measurements of block 122) some anatomical characteristics of the left atrial appendage. Processing of this EP action rule (block 124) is optionally gated by a determination from non-EP data (e.g., positioning information describing the position of an LAAO SHD intervention device as corresponding to a position within the LAA) that the LAAO device “should be” in a correct position, or near to it. In the case of a mismatch between the non-EP data and the EP signal measurements, there is optionally displayed a warning to this effect. In the case of mutual confirmation, there is optionally displayed a confirmation.

TAVI (transcatheter aortic valve implant): In some embodiments, EP signals measured over the course of a procedure (e.g., from a sensing electrode and/or from a TAVI, in the role of the SHD intervention device) optionally include EP signals characteristic of proximity to the bundle of His (and/or optionally any other feature of the heart's electrical conduction system, such as the SA node, the AV node, the crista terminalis, the bundle of Bachman, the right bundle, the left bundle, and/or the Purkinje fiber(s); the bundle of His specifically is described as an example which optionally applies to any of these). The EP signals are optionally accessed (block 120) as they are measured. Optionally, non-EP measurements are accessed (block 122) and used to assign corresponding positions to the EP signals as they are acquired. Accordingly, at the time that actual implantation actions of the TAVI are about to occur, operation of the EP action rule (block 124) can provide action guidance (block 126) that a TAVI implant is not about to be positioned on the bundle of His (e.g., is at a minimal safe distance from it, and/or is oriented to avoid it), where it could possibly exert pressure leading to the complete heart block complication which is associated with TAVI. Provided more particularly as an action guidance display, the action guidance optionally comprises a warning (such as a text, visual indication, and/or tone) when deployment is actually contacting and/or at risk to contact the bundle of His. Additionally or alternatively, the action guidance comprises an annotation (e.g., a tag, alternation in wall color/texture, or another indication) giving the position of the bundle of His on a display of a structural model of the heart.

The same type of guidance is optionally performed for guidance of the implantation of an ASD (atrial septal defect) closure device and/or a mitral and tricuspid annuloplasty ring or band.

Mitral and tricuspid annuloplasty ring or band: in some embodiments, EP signals measured over the course of a procedure are accessed (block 120) and assessed by an EP action rule (block 124) for a relative balance of atrial and/or ventricular electrical function signals. Optionally, positions corresponding to expected and/or previously established EP signals at the level of the valve annulus confirm (block 126) (e.g., before and/or after anchoring) correct identification of the hinges for performing the anchoring of the annuloplasty device.

Optionally, this embodiment of the method of FIG. 1A is performed together with the use of non-EP device position information (block 122). In some embodiments, for example, a structural model of the heart is displayed. Positions at a level along the atrioventricular (AV) axis corresponding to the expected and/or previously established locations of currently measured (e.g., from the SHD intervention device and/or a catheter sheath which deploys it) atrial and/or ventricular electrical function signals are marked. For example, a ring of the heart wall is displayed “lit up” at the level along the AV axis corresponding to the position estimated using the EP signal. Optionally, non-EP position information is presented along with this. For example, a location in 3-D space is marked to identify the separately determined position (e.g., determined using X-ray, ultrasound, electrical field measurements, and/or another method) of the SHD intervention device and/or sheathing catheter. Optionally, any condition of “mismatch” between the EP signal data-derived and non-EP measurement derived position indications is itself flagged, for example, so that corrective action can be taken.

The same type of guidance is optionally performed for guidance of the implantation of a mitral and tricuspid valve clip.

Reference is now made to FIG. 1B, which is a flowchart schematically representing a method of producing guidance for a structural heart disease treatment (e.g., via MISHDI), according to some embodiments of the present disclosure. Embodiments according to FIG. 1B are also embodiments of FIG. 1A, with certain particulars further specified. More specifically, embodiments according to FIG. 1B include use of a heart structural model and localization of recording locations and/or sources of EP signal data within the heart structural model.

At block 130, in some embodiments, a processor accesses one EP signal data from one or more measured EP signals (for example, as described in relation to block 120 of FIG. 1A).

At block 132, in some embodiments, the processor accesses one or more non-EP measurements (for example, as described in relation to block 122 of FIG. 1A). In particular: at block 134, in some embodiments, the processor accesses a structural model of the heart (this is optionally a specific example of the accessing of block 122, of FIG. 1A, wherein accessing of a structural model is optional). The structural model, in some embodiments, corresponds to the “scaffolding” or “basic model” described in relation to the aspect of integration of multimodal measurements. While it is shown specifically associated within non-EP signal data block 132, it optionally is generated at least in part based on previously measured/accessed EP signal data 130.

The structural model optionally based on a primary measurement modality (for example, imaging by electrical field measurements, ultrasound intra-cardiac echocardiography, magnetic field imaging, X-ray, MRI, and/or CT), or built up by the coordinated use of a plurality of measurement methods. Measurements may be made using devices auxiliary to a device which directly provides MISHDI treatment (e.g., imaging probes placed within the heart lumen or at other locations).

At block 136, in some embodiments, the processor localizes the measured EP signals (that is, the associated EP signal data) within the structural model of the heart, using any suitable combination of the measured EP signals and the one or more non-EP measurements. This corresponds to the generation of a “compound model”, with features, for example, as described in relation to the aspect of integration of multimodal measurements.

Methods of localizing EP signal data used in some embodiments of the present disclosure include simultaneous measurement of EP signals while monitoring position of the EP-sensing electrode using another measurement modality. Such other measurement modalities include, for example , electrical field-based sensing, wherein optionally the same electrode senses both EP signals, and impedances associated with one or more electrical fields generated in the same region (e.g., at RF frequencies distinct from the relatively low frequencies associated with EP signals). Additionally or alternatively, probe position is monitored using magnetic field sensing. Additionally or alternatively, EP signals are measured while the measuring probe's position is imaged, for example using echocardiography, X-ray imaging, or another imaging technique. In some embodiments, EP signals themselves comprise positioning data. The “axes” of this positioning data are optionally constructed according to the amplitude and/or timing of a plurality of intracardially measured EP signal components. For example, moving from a right atrium to a right ventricle along the atrioventricular axis, P-wave amplitude tends to decrease while the QRS-wave amplitude tends to increase; signal delays (relative to heart phase) may also increase along this axis. These trends, taken together and/or individually, optionally define a measurement axis approximately corresponding to a linear spatial axis. Heart conduction system structures specialized for signal transmission, such as the SA node and/or bundle of His, have their own characteristic signal characteristics, e.g., waveform shapes and/or delays. Amplitudes of these characteristic EP signal components optionally define a distance constraint on a measuring electrode. As more such constraints are taken into account, the EP signal measurements themselves define a non-spatial coordinate system in which “distances” are defined between different measurement values.

Optionally, this non-spatial coordinate system is converted to an estimated spatial coordinate system using constraints such as the general anatomical organization of heart chambers and/or structures of the heart's electrical conduction system. In some embodiments, a few EP signal measurement values are explicitly anchored to specific spatially specified locations, and locations at intermediate locations (for example) are estimated based on correspondingly intermediate EP signal measurement values.

At block 138, in some embodiments, the processor applies an EP action rule to the one or more measured EP signals and optionally the non-EP measurements. The EP action rule also takes into account the EP signal localization of block 136. Block 138 also corresponds to a more specific implementation of block 124 of FIG. 1A.

EP action rules suitable in particular for use with the method of FIG. 1B are optionally characterized by the use of structure indications both distances and relative direction in a plurality of dimensions (preferably three dimensions, optionally two) between a plurality of elements relevant to the MISHDI. The localization performed in block 136 provides a basis of such indications. Major relevant “elements” in this sense include, for example: the SHD intervention device itself, a target site for implantation of the SHD intervention device, and/or regions to avoid with the SHD intervention device. Other relevant elements optionally include positions of other measurement devices, and positions of heart structural landmarks and/or waypoints. In some embodiments, three or more such elements are simultaneously tracked by inputs and EP action rules (for example, as further described in relation to block 142).

At block 140, in some embodiments, the processor provides action guidance output from the EP action rule applied at block 138 (for example, as described in relation to block 126 of FIG. 1A)

At block 142, in some embodiments, the action guidance output is optionally used to generate an action-guiding display. Optionally, the action-guiding display is created using a structural model of the heart (e.g., the structural model of block 134) and the EP signal localization of block 136. In some embodiments, the action-guiding display is produced according to the flowchart of FIG. 1C.

Reference is now made to FIG. 1C, which is a flowchart schematically representing a method of using rule-generated guidance for a structural heart disease treatment (e.g., via MISHDI) to produce a guidance image, according to some embodiments of the present disclosure.

At block 150, in some embodiments, a processor accesses action guidance output, e.g., a result of blocks 140 and/or 126). Optionally one or more action guidance outputs are accessed.

At block 152, in some embodiments, a processor accesses a structural model of the heart.

At block 154, in some embodiments, a processor produces an action-guiding image of the structural model of the heart, including an indication of the action guidance described by the action guidance output. Optionally, the action-guiding image indication combines a plurality of action guidance outputs.

Action-guiding displays may be explicitly instructive (e.g., providing instructions and/or warning such as “implant/don't implant here/now” and/or “perform pacing validation here/now”), Additionally or alternatively action-guiding displays display procedure context so that what a procedure practitioner is doing (e.g., moving and/or deploying an SHD intervention device) is shown together with elements of concern that should be, or should not be, affected by what the procedure practitioner is doing. In some preferred embodiments, these displays represent spatial reconstructions of the procedure environment, that is, they include a representation of the heart anatomy. In some embodiments, at least portions of the action-guiding display are abstracted from direct spatial representation of the heart anatomy. For example, a meter-type display shows estimated relative position close to or far from a target or region to be avoided (proximity-type meter), optionally with zones marked for conditions of particular relevance such as successful contact (e.g., with target of the SHD intervention device), sufficient proximity (e.g., or dangerous proximity (e.g., of a region to be avoided, and a dangerous proximity that counter-indicates performing of a procedure action using the SHD intervention device such as opening, attachment, or another procedure action).

It is noted that there are optionally a plurality of action-guiding displays that result from a single action guidance output, and conversely that a plurality of action guidance outputs may be collected into a single action-guiding display. Multiple structures may be indicated simultaneously in a spatial representation of a heart, for example. A spatially abstracted (e.g., proximity meter-type) action-guiding display optionally comprises status indicators for a plurality of conditions which update independently, and optionally one or more indicators which indicate a joint status of a plurality of conditions (e.g., both “at the right place” and “not too near any wrong place”).

Examples of action-guiding displays include tagging, coloring, and/or patterning portions of a structural heart model with EP signal data analysis results. For example, inputs comprising certain EP signal components distinguished by waveform characteristics are converted to indications on the structural model showing estimated locations of sources of those EP signal components. In some embodiments, waveforms with different characteristics (e.g., relative amplitudes of components) are assigned to different categories, and shown differently in the image in accordance with category—for example, areas with waveforms characteristic of proximity to structures such as the AV node and the bundle of His are tagged by a visual marker, and/or shown differently (e.g., a different color, brightness, saturation, transparency, and/or texture) than areas with waveforms characteristic of cardiac tissue alone. In some embodiments, a certain ratio of P wave amplitude to QRS complex amplitude (e.g., a ratio of 1:1.5, 1:2, or another ratio) is assigned as indicating a position at the valve, and “midway” along an atrioventricular axis. Optionally, positions at the valve are indicated by a range of ratios, for example, 1:(1.5±0.25), 1:(2±0.25), or another range. Locations at which a larger ratio P wave to QRS complex amplitude is measured are assigned as “atrial”; locations where a relatively smaller ratio is measured are assigned as “ventricular”. embodiments, gradations between two different characteristic waveforms are shown along a color or other visually displayed gradient. For example, conversion along an atrioventricular axis from a dominant (higher-amplitude) P-wave 504 to a dominant QRS complex 503 is shown by different colors selected from a color look-up table.

Optionally, action-guiding display indications are the result of a plurality of inputs integrated by an EP rule. For example, position of an electrode (e.g., of an SHD intervention device, SHD intervention device anchor, and/or SHD intervention device sheath) at a connective tissue valve location is indicated by the joint occurrence of circumstances including: at least approximately correct spatial location (e.g., as measured by an imaging method), impedance measurements indicating constant (e.g., constant as a function of heartbeat cycle) contact with tissue (that, accordingly, is not a moving valve leaflet), EP signal measurements indicating contact with electrically inactive (i.e., non-myocardial) tissue, and/or EP signal measurements indicating that the electrode is not dangerously close to a potentially damage-vulnerable structure of the heart's electrical conduction system such as the bundle of His.

Reference is now made to FIG. 1D, which is a flowchart schematically representing a method of guiding a structural heart disease treatment (e.g., via MISHDI), according to some embodiments of the present disclosure.

At block 102, in some embodiments, a rule which maps spatial position data (e.g., electrical field measurement data, or another source of spatial position data) to positions within a body cavity is accessed.

In some embodiments, the measurements are of mapping electrical fields generated as a plurality of electrical fields which intersect within a body cavity within which a device for performing MISHDI is to be introduced. The measurements are optionally obtained using an electrode probe which is moved within the body cavity. The rule, in some embodiments, provides a transform of electrical field measurements to positions, optionally derived from the electrical field measurements themselves; for example using further constraints such as knowledge of the voltage distributions and/or characteristics of the mapping electrical fields, and/or knowledge about the electrode probe such as inter-electrode distances.

In some embodiments, the rule also defines walls of the body cavity—for example, the rule associates to certain electrical field measurement data a position that has the additional property of being at a wall (lumenal surface) of the body cavity. Optionally, the rule defines positions within tissue volumes beyond the wall surface from the direction of the cavity, for example, cavity wall tissue thicknesses (positions within the tissue of the cavity wall), and/or positions of tissue structures existing beyond the thickness of the body cavity wall.

In some embodiments, the rule operates at least partially on imaging data. Optionally, the imaging data indicates a three-dimensional position of a probe by means of 2-D images (e.g., X-ray images and/or echocardiography images) obtained from different angles, wherein the probe is marked, e.g., by a radiopaque marker, and/or according to its acoustic reflection properties. Optionally, the imaging data indicates a two-dimensional position of a probe, e.g., from a single-angle X-ray image and/or echocardiography image. Information localizing within a third dimension is optionally supplied by knowledge of the image plane (e.g., for echocardiography), and/or auxiliary information such as knowledge of a distance of advance along the atrioventricular axis (e.g., estimated from EP signal measurements, and/or measurement of commanded catheter movements).

At block 104, in some embodiments, there are also accessed heart electrical function measurements associated to the positions of the body cavity which is the mapped-to object of the rule of block 102. In some embodiments, the association comprises knowledge that an electrical function measurement was made at the same place as electrical field (or other spatial location-identifying) measurements which are associated to a certain position of the body cavity by the rule of block 102. In some embodiments, the heart electrical function measurements are associated with further data which may be used in characterization; for example as described in relation to the processing of block 108.

It should be understood that the heart electrical function measurements are optionally measured before and/or after the positioning of the device (at block 106). Optionally, the measurements are made before, during, and/or after a same procedure as is used to position and/or operate the device (at block 106). Measurements from within different procedures are optionally used to establish a pre-procedure baseline, and/or monitor post-procedure effects.

At block 106, in some embodiments, a position of a device used in a minimally invasive structural heart disease intervention (MISHDI) is accessed. The device may be, for example, an implantable device such as an occluder, clip, artificial valve, annuloplasty ring. In some embodiments, the position is determined using measurements from the device itself (e.g., wired with an electrode and/or used as an electrode). In some embodiments, the position is determined using measurements from another device (for example, by imaging); optionally a device such as was used to make the measurements used for generating the rule of block 102.

At block 108, in some embodiments, a computer is used to process the rule, measurements, and position of blocks 102, 104, and 106 to produce an image and/or model which together indicate the relative position of the device of block 106, the positions, including surfaces, of block 102, and, furthermore, estimated positions of electrically active structures, derived from the electrical function measurements of block 104. In some embodiments, positions of electrically active tissue are simply estimated to be where (or about where) the measurement probe was when measurements of heart electrical function activity were taken.

The rule of block 102, together with the accessing of blocks 104 and 106 may be considered together as comprising inputs to a subclass of EP action rules. The computer processing of block 108 may be considered as implementing an EP action rule (e.g., as described in relation to block 124).

In some embodiments, electrical activity at a position is categorized (e.g., as characteristic of a certain type of tissue such as an AV node, phrenic nerve, and/or bundle of His) based on the waveform of the electrical activity recorded, and/or its temporal offset from a reference heart electrical signal waveform. In some embodiments, the reference heart electrical signal is a body surface ECG, or derived from measurements recorded using an intracardially fixed electrode. In some embodiments, a reference electrical signal is provided as an exogenously generated pacing signal, e.g. delivered from an intracardiac stimulating electrode.

Timing and waveform of position-associated electrical activity is then derived from known EP properties, optionally constrained by knowledge of the general anatomical position of the measurement probe at the time the measurement was taken. In some embodiments,

Additionally or alternatively, another EP signal and/or measurement is used, for example, any of the EP signals and/or measurements described herein.

A high resolution position is optionally determined from the data, e.g., to a resolution that associates particular anatomical coordinates with a particular source of electrical function activity. Additionally or alternatively, position is determined more generally to be characteristic of a certain anatomical region. For example, in some embodiments, the electrical activity at the position is compared generally to the waveform expected, in order, e.g., to verify a generally correct anatomical placement of a device within a certain heart chamber or portion thereof; and/or against a certain heart chamber wall.

At block 110, in some embodiments, the image and/or model of block 108 is provided, and/or used to generate guidance for movement and/or operation of the device of block 106. In some embodiments, the image and/or model is presented visually, for example as a 3-D screen image. In some embodiments, guidance comprises an indication that operation of the device of block 106 is safe or not safe; and/or recommended (e.g., to produce a treatment effect) or not recommended. Block 110 may be considered as implementing block 126 of FIG. 1A, or blocks 140, 142 of FIG. 1B. Optionally, guidance image production uses the method of FIG. 1C.

Reference is now made to FIG. 2, which schematically represents implantation of an annuloplasty ring 52 around a tricuspid valve 54 between a right atrium 51 and a right ventricle 55 of a heart 50, according to some embodiments of the present disclosure.

In some embodiments, annuloplasty ring 52 is anchored to tricuspid valve 54 using anchors 53 (applied via catheter 61). There is a risk of accidental damage from an anchor 53 being driven into bundle of His 56, and/or by anchoring annuloplasty ring 52 where it could exert pressure on the bundle of His 56.

In some embodiments, electrodes of an electrode catheter 60 are used to measure positions and electrical activity within at least right atrium 51. In some embodiments, positions where electrical activity of bundle of His 56 is noted are verified to be away from positions of anchors 53 and/or annuloplasty ring 52, before and/or during operations to anchor annuloplasty ring 52.

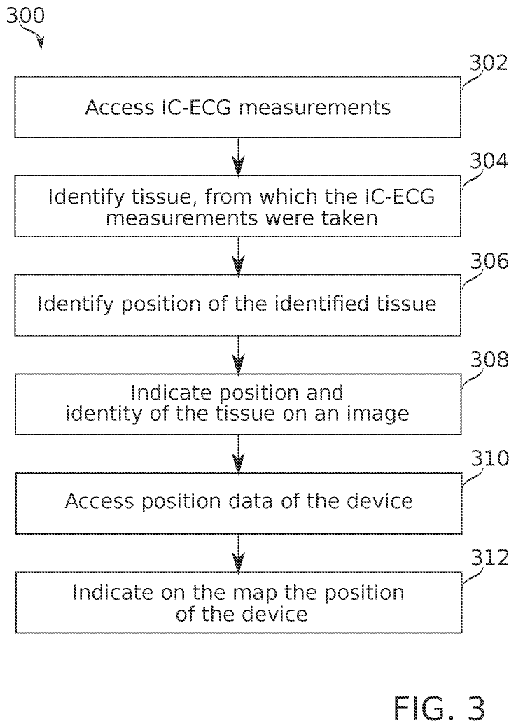

Reference is now made to FIG. 3, which is a flowchart of a method 300 for using

EP signals in the identification of tissue near a measurement device, according to some embodiments of the present disclosure. Method 300 is for indicating positional relationship between heart tissue and a device configured to provide structural heart disease intervention therapy, according to some embodiments.

At 302, measurements made by an in-heart electrode are accessed. The measurements are indicative of electrical activity of heart tissue in the vicinity of the in-heart electrode. The accessed measurements may have been obtained by a mapping catheter, introduced into a heart chamber, in which the intervention is planned to take place. The mapping catheter may be used for generating an image of the heart chamber, for example, as described in International Patent Publication No. WO2018/130974, in International Patent Publication No. WO2019/034944, and/or in International Patent Publication No. WO2019/035023. Alternatively, the mapping catheter may be tracked by any other tracking method, and the tracked position of the catheter may be registered to an image of the heart by any suitable registration method known in the art. For example, the image of the heart may be a CT of the patient, and the registration method may be that described in International Patent Publication No. WO2018/078540, the contents of which are included herein by reference in their entirety. Whether the mapping catheter is used for generating a map or not, it is used for measuring intracardiac electrocardiogram data (IC-ECG data). In some embodiments, the IC-ECG data measured are recorded associated with the position the mapping catheter (or the ECG reading electrode of the mapping catheter) had when the IC-ECG data were measured.

At 304, the heart tissue, from which IC-ECG data were measured, are identified based on the IC-ECG measurements. For example, in some embodiments, IC-ECG data are recorded simultaneously with surface body ECG data, and the tissue is identified based on a comparison between the IC- and BS-ECG data.

At 306, positions of different heart tissues from which IC-ECG data were measured, are identified. The positions identified at 306 may be of tissue identified at 304. The position identification may be, for example, based on data indicative of the position of the in-heart electrode, recorded when the in-heart electrode measured the electrical activity of the heart tissue. In some embodiments, such data is accessed, and the position of the identified tissue is identified based on the accessed data.

At 308, the position and identification of one or more heart tissues are indicated on an image of the heart. The image of the heart may be one generated during the procedure, or one generated before the procedure. Optionally, the image is an image from an atlas. The positions identified for the tissues are indicated on the image, for example, by registering the position data to the image. The identifications of the different tissues is also indicated on the image, for example, by coloring different tissues with different colors. For example, ventricles may be colored by blue, atria by green, the AV node by purple, and the bundle of His by red.

At 310, measurements indicative of a position of the device configured to provide structural heart disease intervention therapy are accessed. The position of the device may be measured using any tracking system known in the art per se, and may be registered with the image by any suitable registration method. In some embodiments, the mapping electrodes may have generated a rule to transfer electrical measurements of fields generated by other electrodes (body surface or in-body electrodes, located at known positions), to positions, for example, as described in International Patent Publication No. WO2019/034944. In some such embodiments, the device is used for measuring electrical measurements of the same fields, and the rule is used for transferring these electrical measurements to positions. In some embodiments, the device itself may be used as an electrode for measuring the electrical measurements. Alternatively, one or more electrodes may be attached to the device, and used for the measurements.

At 312, the position of the device is indicated on the map, on which the location and identification of one or more heart tissues is also indicated. For example, the position of the device may be indicated by an icon having a shape and size similar to those of the device, and the icon may be positioned in the image, centered at the location identified for the device. Optionally, the physician may take decisions based on the image, for example, the physician may decide not to anchor an implant in a way that exerts pressure on the bundle of His, to prevent damaging the auto-pacing of the patient's heart.

### System for Compound Model-Guidance of SHD Interventions Using EP Signal Data

Reference is now made to FIG. 4, which schematically represents a block diagram of a system 400 configured to use a compound model incorporating EP signal data to guide SHD interventions, according to some embodiments of the present disclosure.

In overview: Block 401 corresponds to EP signal data (mentioned by type), and block 410 corresponds to non-EP data (mentioned by type). Any particular sub-block of blocks 401, 410 is optional, depending on the particular system configuration used. Actual devices which measure, acquire, or otherwise generate the data of blocks 401, 410 are optionally part of an embodiment of system 400 itself, and optionally are provided separately. The individual sub-blocks of blocks 401, 410 are labeled in FIG. 4 according to the type of the data accessed by the system 400. The data is generated, for example, as further related to hereinbelow. Block 420 corresponds to a computer processing system, including functional modules programmed and otherwise configured to access and process the data of blocks 401, 410 as described elsewhere herein, and as related to more specifically hereinbelow. Block 430 corresponds to a display or other output device used to present guidance, for example in the form of images and/or text and/or symbolically communicated messages.

As noted by the sub-blocks of EP signal data block 401, examples of electrophysiological data include:

IC-ECG data 402; measured, for example by an electrode positioned at an intracardiac location. The IC-ECG may be unipolar or bipolar. In some embodiments, the bipolar IC-ECG signals are measured between two electrodes of an in-body catheter (for example, a lasso catheter), or any other electrode known from the field of electrophysiology. Preferably, the catheter carries a plurality of electrodes, to allow bipolar IC-ECG measurements, and optionally, to allow imaging as described, for example, in International Patent Publication No. WO2018/130974, in International Patent Publication No. WO2019/034944, and/or in International Patent Publication No. WO2019/035023. Optionally, recording from an electrode positioned at another intra-body position, close enough to the heart that it receives location-specific electrophysiological signals is used instead of/additionally to intracardiac measured ECG data.

BS-ECG data 403, recorded from any suitable arrangement of body surface electrodes and/or “leads” (e.g., 12-lead or 3-lead; leads correspond to actual single electrodes, and/or combinations of electrodes acting as a “virtual” electrode).

EP signal data of either block optionally include measurements of either or both of intrinsic electrical activity, and induced (e.g., paced) electrical activity.

As noted within the sub-blocks of non-EP data block 410, optional blocks 411-418 are labeled with the data types provided, which in turn correspond more particularly to data types:

Providing image-type heart structure information 411 (e.g., electrical field-based mapping/imaging, magnetic field-based mapping/imaging, echocardiography imaging, X-ray imaging, CT, and/or MRI). This information is optionally provided in the form of a “structural model” of a heart; that is, an anatomical reconstruction of the heart based on one or more imaging/mapping data sources.

Providing intracardiac probe localization information 416 (e.g., of an electrode, magnet, or other detector/emitter placed on a probe). This optionally includes, for example, probe position, movements, and/or speeds. The localization information may be provided initially already in a frame of reference specified by the heart structure information, or there may be other information made use of to register the probe localization information with the heart structure information.

Providing other measurement apparatus configuration information 412 and/or SHD intervention device configuration information 415 (e.g., placement and/or electrical characteristics of body surface electrodes, relative distances of intracardiac electrodes, dimensions and optionally other mechanical operating characteristics of SHD intervention devices and/or other tools used in the MISHDI procedure). Other mechanical operating characteristics include, for example, stiffness, bending radius, and/or deployment state (e.g., shape) as a function of deployment command/stage (e.g., advancement of a device out of a constraining sheath).

Providing other SHD intervention device/procedure tool/procedure status data 413. In some embodiments, this includes information from SHD intervention devices and/or other tools which are themselves equipped with sensors and/or encoders which measure information such as the relative positioning of mechanically operated control members, and/or forces exerted on the SHD intervention device/tool. In some embodiments, this includes data describing administration of a chemical compound such as saline, contrast agent, or a pharmacological agent. The data may include, for example: amounts/concentrations, start time, and/or stop time.

Providing user inputs 417 (e.g., user interface input via pointer device and/or keyboard, voice input, gesture input, or another method). User inputs are used for example: to set and/or confirm an operational mode within which measurements of other types are interpreted and/or integrated, to provide parameters (e.g., patient identification and/or vital statistics data), to select from among options, and/or to select from among and/or tune display modes and/or appearances.

Providing information from already-implanted devices 414 which act as an “input” to heart EP function (e.g., pacemaker and defibrillator outputs and/or parameters).

Providing non-EP measures of physiological function 418; for example, one or more of:
Blood flow volume, velocity and/or direction, for example, as measured through a voxel and/or volume of interest via Doppler echocardiography or another method.
Blood pressure—for example, systemic, intravascular, intracardiac; and, for example, absolute or differential between two or more locations.
Heart sounds.
Parameters of breathing such as rate, amplitude, and/or total volume.
Blood conductivity.

Computer processing system 420, in some embodiments, is programmed to include a structure tracking module 421, location tracking module 422, rule calculating module 423, EP signal interpreting module 424, and/or guidance generator 425.

Structure tracking module 421, in some embodiments, is programmed to access one or more of image-type heart structure data 411, and intracardiac electrode localization data 416, in order to receive, generate, update, and/or maintain a structural representation of the heart in which the SHD intervention occurs. The structural representation is optionally an image of heart structure (2-D or 3-D), to which other data is registered (e.g., via location tracking module 422), but which is not necessarily itself segmented or otherwise processed to produce a structural model.

In some embodiments, the structural representation encodes geometry of the heart, including in particular spatially detailed representations of heart wall locations; and optionally including spatially detailed heart structures such as heart valves (and optionally their individual components such as leaflets and/or chordae), fossae, ostia, appendages, heart electrical conduction system structures, and/or blood vessels (in their appropriate places). Any of these optionally is statically represented, or represented as varying as a function of time. A “spatially detailed” representation of a heart, for purposes of embodiments of the present disclosure, represents structure by defining surfaces adjusted to represent heart features through a geometrical extent corresponding generally to their anatomical shape and size—as compared, for example, to an abstraction like a sphere, box, or tag. A structural representation of a heart is, moreover, adjusted to the particular heart of a given MISHDI procedure, at least insofar as is needed to present guidance relying on spatial relationships like distance among cardiac features of particular relevance to the current procedure, wherein the guidance takes into account the specific anatomy of the heart, e.g., to find and/or avoid a structure of the heart. The structural representation may be encoded, for example, as an image (e.g., a segmented 3-D image) and/or as a 3-D structural model, e.g., as a mesh of polygons representing surface locations. Additionally or alternatively, in some embodiments, the structural representation comprises a mapping of measurements and/or properties to positions.

In some embodiments, a structural representation is converted to a form suitable for display; e.g., a digital image in a format compatible with a computer display monitor. Herein, it should be understood that phrases such as “displaying a structural representation” and “presenting a display” includes use of such conversions as appropriate. A display form of a structural representation is optionally partial and/or schematic.

In some embodiments, structural tracking module 421 accesses an already existing structural representation (e.g., structural model) of the heart, e.g., as provided by image-type heart structure data 411; accessing the data as raw images and/or as already segmented and/or otherwise processed to provide a structural model. In some embodiments, structural tracking module 421 is configured to itself generate the structural model from image data (which is one form of anatomical reconstruction—generating a model of the anatomy, based on, e.g., segmentation of image data, and optionally recognition of segmented portions of the image data). Additionally or alternatively, structural tracking module 421 generates a structural model using intracardiac probe localization data 416, for example as described in International Patent Publication No. WO2018/130974 in International Patent Publication No. WO2019/034944, and/or in International Patent Publication No. WO2019/035023, the contents of which are included herein by reference in their entirety. Generation of a structural model using intracardiac probe localization data another form of anatomical reconstruction the anatomy of, e.g., a lumen is reconstructed based on places visited within the lumen, and/or on measurements made at the places visited in addition to measurements which localize the probe. Optionally, structural tracking module 421 receives such a structural model already created.

In some embodiments, structural tracking module 421 continues to refine and/or extend the structural model as new data is received, e.g., from intracardiac probe localization.

Location tracking module 422, in some embodiments, comprises a module which tracks locations of devices, including probes which make measurements, as they move about within the heart represented/modeled by structure tracking module 421. The localized device can be any probe used in the procedure—for example, an electrode catheter, the SHD intervention device itself, and/or a delivery sheath used to advance the SHD intervention device into position and/or to deploy the SHD intervention device.

In some embodiments, location tracking module 422 allows information provided from EP signal interpreting module(s) 424 to be assigned to particular locations within the structural representation/model maintained by structure tracking module 421. For example, EP signal data processed by EP signal interpreting module(s) 424 is assigned a location in the structural representation/model, based on tracking by location tracking module 422 of the probe that measured the EP signal. Location tracking module 422 operates by using any available position information. In some embodiments, this comprises in particular intracardiac probe localization data 416. Optionally, other data is used to assist in localization; for example EP signal data itself may be at least partially indicative of location, e.g., position along an atrioventricular axis, depending on relative amplitudes of P and QRS waves.

Optionally, one or more EP signal interpreting modules 424 are provided, which allow EP signals to be analyzed to more particular aspects of the EP signal, for example based on waveform shape, latency, and/or amplitude.

The result of a given aspect of the EP signal in terms of procedure guidance is provided by one or more rule calculating module(s) 423, which optionally accepts inputs as direct input from EP signal data 401, non-EP data 410, and/or as input processed from one of the other processing module types 421, 422, 424. The rules applied by rule calculating module(s) 423 correspond to the “action rules” described, for example, in relation to FIG. 1A, herein.

In some embodiments, guidance generator 425 generates guidance from the output of rule calculating module(s) 423, optionally also using data from structural tracking module 421 (e.g., in order to generate an image of the heart within which the procedure is taking place), and/or location tracking module 422 (e.g., to allow showing a current position of one or more probes within the heart). As various locations within the heart are identified, for example based on EP signal data/or non-EP data, they are optionally also added to the image of the heart (all on a single image, or optionally divided among different images showing different aspects of heart structure and/or function). Identification may be, for example, as particular anatomical structures and/or as regions having particular EP signal characteristics. Additionally or alternatively, in some embodiments, guidance generator 425 generates guidance from raw inputs. For example, in some embodiments, direct graphs of EP signal data 401 are presented by guidance generator 425. Generation of action guidance is described, for example, in relation FIG. 1C, herein.

Display 430, in some embodiments, is used to present action guidance to an operator. Guidance can be of different types. Structure-type guidance shows a user where portions of the heart are—e.g., the shape of the heart, the location of anatomical and/or functional features within the heart. Tracking-type guidance shows where a component being used in a procedure is, whether tracked using EP signal data or non-EP data. In some embodiments, more direct action guidance is provided. For example, particular meaning is associated (e.g., via action rules) with inputs received and/or a current state of the procedure, and guidance issued accordingly. Action guidance optionally comprises, for example, warnings, targets, and/or images; and/or procedural instructions to help an operator avoid, achieve, verify and/or mitigate conditions of the procedure.

### General

As used herein with reference to quantity or value, the term “about” means “within ±10% of”.

The terms “comprises”, “comprising”, “includes”, “including”, “having” and their conjugates mean: “including but not limited to”.

The term “consisting of” means: “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

The words “example” and “exemplary” are used herein to mean “serving as an example, instance or illustration”. Any embodiment described as an “example” or “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the present disclosure may include a plurality of “optional” features except insofar as such features conflict.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

Throughout this application, embodiments may be presented with reference to a range format. It should be understood that the description in range format is merely for convenience and brevity and should not be construed as an inflexible limitation on the scope of descriptions of the present disclosure. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Although descriptions of the present disclosure are provided in conjunction with specific embodiments, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present disclosure. To the extent that section headings are used, they should not be construed as necessarily limiting.

It is appreciated that certain features which are, for clarity, described in the present disclosure in the context of separate embodiments, may also be provided in combination in a single embodiment. Conversely, various features, which are, for brevity, described in the context of a single embodiment, may also be provided separately or in any suitable subcombination or as suitable in any other described embodiment of the present disclosure. Certain features described in the context of various embodiments are not to be considered essential features of those embodiments, unless the embodiment is inoperative without those elements.

In addition, any priority document(s) of this application is/are hereby incorporated herein by reference in its/their entirety.

1-18. (canceled)

19. A method of guiding a procedure for implanting a structural heart disease intervention device in a heart, the method comprising:
accessing a baseline value of a parameter of an electrophysiological (EP) signal measurement;
accessing EP signal measurements obtained after an action performed using the device, the action being performed upon a heart wall and/or a heart valvular apparatus;
processing, using a computer processor, the EP signal measurements to identify a change of the parameter from the baseline value; and
presenting guidance for a next action to be performed in the procedure for implanting the structural heart disease intervention device, based on the identified change.

20. The method of claim 19, wherein the action affects a mechanical and/or hemodynamic function of the heart.

21. The method of claim 19, wherein the processing comprises estimating a location of the heart having changed electrophysiological activity resulting in the identified change.

22. The method of claim 21, wherein the presenting comprises displaying a structural representation of the heart including an indication of a location on the structural representation corresponding to the location of the heart having changed electrophysiological activity resulting in the identified change.

23. The method of claim 19, wherein the identified change comprises a change in at least one of the group consisting of:
a waveform morphology,
a waveform amplitude,
a waveform duration,
a waveform frequency,
a waveform slope, and
a spectral content of an EP signal.

24. The method of claim 19, wherein the changed parameter comprises at least one of the group consisting of:
EP signal rate,
EP signal rhythm,
variability in EP signal rhythm,
QRS duration,
QRS spectral content,
P wave morphology,
PR interval,
ST segment, and
T wave morphology.

25. The method of claim 19, wherein the changed parameter comprises a conduction time measured for at least one of the group consisting of conduction time between:
electrodes,
locations,
EP signal components, and
delivery of a pacing signal and a signal arrival at a location.

26. The method of claim 19, wherein the changed parameter comprises an intracardially measured local activation time of a first location within the heart, relative to at least one of the group consisting of:
a phase of a BS-ECG,
a local activation time of a second location within the heart measured intracardially, and
a previously measured intracardially measured local activation time of the first location within the heart.

27. The method of claim 19, wherein the processing comprises determination of a change in a result of a pacing test, or a change in a measurement value with and without pacing.

28. The method of claim 19, wherein the identified change indicates potential damage to a heart electrical system structure, and the guidance comprises instruction to reverse an implantation of the structural heart disease intervention device.

29. The method of claim 19, wherein the identified change indicates a level of isolation between components of the heart electrical system, and the guidance comprises an indication as to whether a targeted level of isolation has been achieved.

30. The method of claim 19, wherein the accessed electrophysiological measurements indicating electrical activity of tissue of the heart comprise measurements of an electrophysiological study described by a CPT code.

31. A system comprising a processor, memory, and display, wherein the memory is configured with instructions instructing the processor to:
access a baseline value of a parameter of an electrophysiological (EP) signal measurement;
access EP signal measurements obtained after an action performed within the heart which modifies at least a mechanical and/or hemodynamic function of the heart;
process the EP signal measurements to identify a change of the parameter from the baseline value; and
present, using the display, guidance for a next action to be performed within the heart, wherein the guidance presented is based on the identified change.

32. The system of claim 31, wherein the EP signal measurements are obtained using an electrode within the heart, and the guidance guides a next action performed using a structural heart disease treatment device positioned within the heart.

33. The system of claim 31, wherein the processor is further instructed to estimate a position and/or identity of a location in the heart having changed electrophysiological activity resulting in the identified change.

34. The system of claim 33, wherein the guidance presented comprises a structural representation of the heart including an indication of a position and/or identity of a location of the heart having changed electrophysiological activity resulting in the identified change.

35. The system of claim 31, wherein the processor is instructed to identify a change in at least one of the group consisting of:
an EP signal waveform morphology,
an EP signal waveform amplitude,
an EP signal waveform duration,
an EP signal waveform frequency,
an EP signal waveform slope, and
a spectral content of an EP signal.

36. The system of claim 31, wherein the changed parameter comprises at least one of the group consisting of:
EP signal rate,
EP signal rhythm,
variability in EP signal rhythm,
QRS duration,
QRS spectral content,
P wave morphology,
PR interval,
ST segment, and
T wave morphology.

37. The system of claim 31, wherein the changed parameter comprises a conduction time measured for at least one of the group consisting of conduction time between:
electrodes,
locations,
EP signal components from signal sources located along a transmission pathway, and
delivery of a pacing signal and a signal arrival at a location.

38. The system of claim 31, wherein the changed parameter comprises an intracardially measured local activation time of a first location within the heart, relative to at least one of the group consisting of:
a phase of a BS-ECG,
a local activation time of a second location within the heart measured intracardially, and
a previously measured intracardially measured local activation time of the first location within the heart.

39. The system of claim 31, wherein the EP signal measurements are processed to determine a change in a result of a pacing test, or a change in a measurement value with and without pacing.

40. The system of claim 31, wherein the identified change indicates potential damage to a heart electrical system structure, and the guidance comprises instruction to reverse an implantation of the structural heart disease intervention device.

41. The system of claim 31, wherein the identified change indicates a level of isolation between components of the heart electrical system, and the guidance comprises an indication as to whether a targeted level of isolation has been achieved.

42-84. (canceled)

What is claimed is:

1. A method of guiding a procedure for implanting a structural heart disease intervention device, the method comprising:
accessing a structural representation of a portion of a heart;
accessing electrophysiological measurements indicating electrical activity of tissue of the heart;
accessing position measurements indicating a position of the device within the portion of the heart;
processing the electrophysiological measurements to identify a location of a heart structure within the portion of the heart; and
presenting the heart structure location, relative to the position of the device; wherein the electrophysiological measurements and the position measurements are obtained during the procedure being guided.

2. The method of claim 1, wherein the presenting comprises presenting a display showing the portion of the heart, a position of the device within the portion of the heart, and an indication of the position of the heart structure within the portion of the heart.

3. The method of any one of claims 1-2, wherein the processing comprises production of a structural model of the portion of the heart wherein the heart structure location is represented as a characteristic of a portion of the structural model.

4. The method of any one of claims 1-3, wherein the presenting comprising presenting a warning that the heart structure location is in a dangerous proximity to the device.

5. The method of claim 4, wherein the warning instructs not performing a procedure action.

6. The method of any one of claims 1-3, wherein the presenting comprising presenting a confirmation that the heart structure location is at a sufficient distance from the device.

7. The method of claim 6, wherein the confirmation instructs a procedure action is allowed.

8. The method of any one of claims 1-3, wherein the presenting comprising presenting a confirmation that the heart structure location is in a sufficient proximity to the device.

9. The method of claim 8, wherein the confirmation instructs performing a procedure action.

10. The method of any one of claims 1-3, wherein the presenting comprising presenting a warning that the heart structure location is in insufficient proximity to the device.

11. The method of claim 4, wherein the warning instructs not performing a procedure action.

12. The method of any one of claims 5, 7, 9, and 11, wherein the procedure action comprises at least one from the group consisting of:
attaching the device
expanding the device,
contracting the device,
retrieving the device,
at least partially de-implanting the device,
penetrating tissue with the device,
pacing heart electrical activity using the device, and
measuring heart electrical activity using the device.

13. The method of any one of claims 1-12, wherein the structural heart disease intervention device is a device selected from the group consisting of:
a left atrial appendage occluder,
a transcatheter aortic valve implant,
a mitral valve clip,
a tricuspid valve clip,
a mitral annuloplasty ring or band,
a tricuspid annuloplasty ring or band, and
an atrial septal defect closure device.

14. The method of any one of claims 1-9, wherein the position measurements indicating the position of the device comprise electrical field measurements made using an electrode of the device.

15. The method of any one of claims 1-9, wherein the position measurements indicating the position of the device comprise EP measurements made using an electrode of the device.

16. The method of any one of claims 1-15, wherein the EP measurements are made using an electrode of the device.

17. The method of any one of claims 1-15, wherein the EP measurements are made using an electrode of the device to pace the electrical activity of tissue of the heart.

18. The method of any one of claims 1-17, wherein the structural representation is a three-dimensional structural representation.

19. A method of guiding a procedure for implanting a structural heart disease intervention device in a heart, the method comprising:
accessing a baseline value of a parameter of an EP signal measurement;
accessing EP signal measurements obtained after an action performed using the device, the action being performed upon a heart wall and/or a heart valvular apparatus;
processing, using a computer processor, the EP signal measurements to identify a change of the parameter from the baseline value; and
presenting guidance for a next action to be performed, based on the identified change.

20. The method of claim 19, wherein the action affects a mechanical and/or hemodynamic function of the heart.

21. The method of claim 19, wherein the processing comprises estimating a location of the heart having changed electrophysiological activity resulting in the identified change.

22. The method of claim 21, wherein the presenting comprises displaying a structural representation of the heart including an indication of the location of the heart having changed electrophysiological activity resulting in the identified change.

23. The method of claim 19, wherein the identified change comprises a change in at least one of the group consisting of:
a waveform morphology,
a waveform amplitude,
a waveform duration,
a waveform frequency,
a waveform slope, and
a spectral content of an EP signal.

24. The method of any one of claims 19-23, wherein the changed parameter comprises at least one of the group consisting of:
EP signal rate,
EP signal rhythm,
variability in EP signal rhythm,
QRS duration,
QRS spectral content,
P wave morphology,
PR interval,
ST segment, and
T wave morphology.

25. The method of any one of claims 19-24, wherein the changed parameter comprises a conduction time measured for at least one of the group consisting of conduction time between:
electrodes,
locations,
EP signal components, and
delivery of a pacing signal and a signal arrival at a location.

26. The method of any one of claims 19-25, wherein the changed parameter comprises an intracardially measured local activation time of a first location within the heart, relative to at least one of the group consisting of:
a phase of a BS-ECG,
a local activation time of a second location within the heart measured intracardially, and
a previously measured intracardially measured local activation time of the first location within the heart.

27. The method of any one of claims 19-23, wherein the processing comprises determination of a change in a result of a pacing test, or a change in a measurement value with and without pacing.

28. The method of any one of claims 19-27, wherein the identified change indicates potential damage to a heart electrical system structure, and the guidance comprises instruction to reverse an implantation of the structural heart disease intervention device.

29. The method of any one of claims 18-27, wherein the identified change indicates a level of isolation between components of the heart electrical system, and the guidance comprises an indication as to whether a targeted level of isolation has been achieved.

30. The method of any one of claims 18-29, wherein the accessed electrophysiological measurements indicating electrical activity of tissue of the heart comprise measurements of an electrophysiological study described by a CPT code.

31. A system comprising a processor, memory, and display, wherein the memory is configured with instructions instructing the processor to:
access a baseline value of a parameter of an EP signal measurement;
access EP signal measurements obtained after an action performed within the heart which modifies at least a mechanical and/or hemodynamic function of the heart;
process the EP signal measurements to identify a change of the parameter from the baseline value; and
present, using the display, guidance for a next action to be performed within the heart, wherein the guidance presented is based on the identified change.

32. The system of claim 31, wherein the EP signal measurements are obtained using an electrode with the heart, and the guidance guides a next action performed using a structural heart disease treatment device positioned within the heart.

33. The system of any one of claims 31-32, wherein the processor is further instructed to estimate a position and/or identity of a location of the heart having changed electrophysiological activity resulting in the identified change.

34. The system of claim 33, wherein guidance presented comprises a structural representation of the heart including an indication of a position and/or identity of a location of the heart having changed electrophysiological activity resulting in the identified change.

35. The system of any one of claims 31-34, wherein the processor is instructed to identify a change in at least one of the group consisting of:
an EP signal waveform morphology,
an EP signal waveform amplitude,
an EP signal waveform duration,
an EP signal waveform frequency,
an EP signal waveform slope, and
a spectral content of an EP signal.

36. The system of any one of claims 31-35, wherein the changed parameter comprises at least one of the group consisting of:
EP signal rate,
EP signal rhythm,
variability in EP signal rhythm,
QRS duration,
QRS spectral content,
P wave morphology,
PR interval,
ST segment, and
T wave morphology.

37. The system of any one of claims 31-36, wherein the changed parameter comprises a conduction time measured for at least one of the group consisting of conduction time between:
electrodes,
locations,
EP signal components from signal sources located along a transmission pathway, and
delivery of a pacing signal and a signal arrival at a location.

38. The system of any one of claims 31-37, wherein the changed parameter comprises an intracardially measured local activation time of a first location within the heart, relative to at least one of the group consisting of:
a phase of a BS-ECG,
a local activation time of a second location within the heart measured intracardially, and
a previously measured intracardially measured local activation time of the first location within the heart.

39. The system of any one of claims 31-37, wherein the EP signal measurements are processed to determine a change in a result of a pacing test, or a change in a measurement value with and without pacing.

40. The system of any one of claims 31-39, wherein the identified change indicates potential damage to a heart electrical system structure, and the guidance comprises instruction to reverse an implantation of the structural heart disease intervention device.

41. The system of any one of claims 31-39, wherein the identified change indicates a level of isolation between components of the heart electrical system, and the guidance comprises an indication as to whether a targeted level of isolation has been achieved.

42. A method of guiding a procedure for implanting a structural heart disease intervention device in a heart, the method comprising:
accessing reference EP signal measurements;
accessing identifying EP signal measurements comprising a signal component characteristic of a location within the heart;
processing, using a computer processor, the reference and identifying EP signal measurements to identify the location, based on timing of an event in the identifying EP signal measurements, relative to events in the reference EP signal measurements; and
presenting guidance for a next action to be performed, based on the identified location.

43. The method of claim 42, wherein the identifying EP signal measurements comprise an IC-ECG measured at a first location within the heart, and the reference EP signal measurements comprise an IC-ECG measured at a second location within the heart.

44. The method of claim 42, wherein the reference EP signal measurements comprise a BS-ECG, and the identifying EP signal measurements comprises an IC-ECG.

45. The method of any one of claims 42-44, wherein the processing comprises identifying a source location of an EP signal measured by the identifying EP signal measurements as comprising a first particular heart electrical system structure, based on comparison to reference EP signal measurements comprising at least one of the group consisting of: an electrogram associated with a second particular heart electrical system structure, and a previously measured electrogram associated with the first particular heart electrical system structure.

46. The method of any one of claims 42-45, wherein the accessed EP signal measurements comprise measurements of an electrophysiological study described by a CPT code.

47. A system comprising a processor, software, and display, wherein the memory is configured with instructions instructing the processor to:
access reference EP signal measurements;
access identifying EP signal measurements characteristic of a location within a heart;
process the reference and identifying EP signal measurements to identify the location, based on timing of an event in the identifying EP signal measurements, relative to events in the reference EP signal measurements; and
present guidance for a next action to be performed, based on the identified location.

48. The system of claim 47, wherein the identifying EP signal measurements comprise an IC-ECG measured at a first location within the heart, and the reference EP signal measurements comprise an IC-ECG measured at a second location within the heart.

49. The system of claim 47, wherein the reference EP signal measurements comprise a BS-ECG, and the identifying EP signal measurements comprise an IC-ECG.

50. The system of any one of claims 47-49, wherein the processor is instructed to identify a source location of an EP signal measured by the identifying EP signal measurements comprising a first particular heart electrical system structure, based on comparison to reference EP signal measurements comprising at least one of the group consisting of: an electrogram associated with a second particular heart electrical system structure, and a previously measured electrogram associated with the first particular heart electrical system structure.

51. A method of guiding a medical procedure within a body cavity comprising:
accessing a rule, wherein the rule maps electrical field measurements to body cavity positions;
accessing measurements of heart electrical function, the measurements being associated to the body cavity positions;
accessing data indicative of a position within the body cavity of a device used to provide structural heart disease intervention therapy;
using the rule, the measurements of heart electrical function, and the position to produce an image and/or model indicating a feature of heart electrical functioning at the position; and
providing guidance indicating device position relative to features of the image and/or model, including in relation to one or more signal sources of the measurements of heart electrical function.

52. The method of claim 51, wherein the measurements of heart electrical function are associated to the body cavity positions by measurement from a same probe at a same position of the probe.

53. The method of claim 51, wherein the measurements of heart electrical function are associated to the body cavity positions by measurement from a same probe during a same time.

54. The method of any one of claims 51-53, wherein the measurements of heart electrical function indicate activity of a bundle of His.

55. The method of any one of claims 51-53, wherein the measurements of heart electrical function indicate activity of an AV node.

56. The method of any one of claims 51-53, wherein the measurements of heart electrical function indicate activity of a phrenic nerve.

57. The method of any one of claims 51-56, wherein the device comprises one of:
an annuloplasty ring;
a heart valve clip;
a left atrial appendage occluder;
a ventricular septal defect clip;
a heart valve implant; and
a heart valve replacement.

58. The method of any one of claims 51-57, wherein the position of the device is measured using the device as an electrode.

59. The method of any one of claims 51-57, wherein the position of the device is measured using an electrode probe in contact with the device.

60. The method of any one of claims 51-59, wherein the measurements of heart electrical function are made along with measurement of a reference signal, and the using comprises comparing the reference signal to the heart electrical signal measurements.

61. The method of any one of claims 51-60, wherein the accessed electrophysiological measurements indicating electrical activity of tissue of the heart comprise measurements of an electrophysiological study described by a CPT code.

62. A system comprising a processor, memory, and display, wherein the memory is configured with instructions instructing the processor to:
access a rule, wherein the rule maps electrical field measurements to body cavity positions;
access measurements of heart electrical function, the measurements being associated to the body cavity positions;
access data indicative of a position within the body cavity of a device used to provide structural heart disease intervention therapy;
use the rule, the measurements of heart electrical function, and the position to produce an image and/or model indicating a feature of heart electrical functioning at the position; and
provide, using the display, guidance indicating device position relative to features of the image and/or model, including in relation to one or more signal sources of the measurements of heart electrical function.

63. The system of claim 62, wherein the measurements of heart electrical function are associated to the body cavity positions by measurement from a same probe at a same position of the probe.

64. The system of claim 62, wherein the measurements of heart electrical function are associated to the body cavity positions by measurement from a same probe during a same time.

65. The system of any one of claims 62-64, wherein the device comprises one of:
an annuloplasty ring;
a heart valve clip;
a left atrial appendage occluder;
a ventricular septal defect clip;
a heart valve implant; and
a heart valve replacement.

66. The system of any one of claims 62-65, wherein the measurements of heart electrical function are made along with measurement of a reference signal, and processor compares the reference signal to the heart electrical signal measurements to produce the image and/or model.

67. A method of guiding a medical procedure within a body cavity, comprising:
accessing measurements of heart electrical function, the measurements being associated to positions within the body cavity;
accessing data indicative of a position within the body cavity of a device used to provide structural heart disease intervention therapy;
using the measurements of heart electrical function, their positions, and the position of the device to determine whether the device and one or more signal sources of the measurements of heart electrical function coincide; and
providing guidance indicating device position in relation to the one or more signal sources of the measured heart electrical function.

68. The method of claim 67, wherein the heart electrical field measurements are associated to the body cavity positions by measurement from a same probe at a same position of the probe.

69. A method of guiding a procedure to intervene in heart structural disease, comprising:
selecting to do a procedure to intervene in a heart structural disease, wherein the procedure is associated with a potential complication comprising damage to a critical structural feature of heart electrical function;
mapping electrical function in at least the critical structural feature using a same tool used to guide positioning within the procedure to produce a map of electrical function; and
performing the procedure using the tool and the map of electrical function.

70. A method of indicating positional relationship between an identified heart tissue and a device configured to provide structural heart disease intervention therapy, the method comprising:
accessing measurements, made by an in-heart electrode, and indicative of electrical activity of heart tissue in the vicinity of the in-heart electrode;
identifying the heart tissue based on the measurements;
identifying a position of the heart tissue based on data indicative of the position of the in-heart electrode when the in-heart electrode measured the electrical activity of the heart tissue;
accessing measurements indicative of a position of the device; and
indicating, on an image of the heart, the position identified for the heart tissue, the identification of the heart tissue, and the position of the device.

71. The method of claim 70, wherein the heart tissue is identified as a tissue that should not be intervened with by the device.

72. The method of claim 70, wherein the heart tissue is identified as the AV node.

73. The method of claim 70, wherein the heart tissue is identified as the bundle of His.

74. The method of claim 70, wherein the heart tissue is identified as one of an artery or a ventricle.

75. The method of any one of claims 70 to 74, wherein the heart tissue is identified based on comparison of ECG signals measured by the in-heart electrode during some time period, and surface ECG signals measured during the same time period.

76. The method of any one of claims 70-75, wherein the device comprises one of:
an annuloplasty ring;
a heart valve clip;
a left atrial appendage occluder;
a ventricular septal defect clip;
a heart valve implant; and
a heart valve replacement.

77. The method of any one of claims 70-76, wherein the position of the device is measured using the device or a portion thereof as an electrode.

78. The method of any one of claims 70-76, wherein the position of the device is measured using an electrode probe in contact with the device.

79. The method of any one of claims 70 to 78, wherein the measurements made by the in-heart electrode are measurements of intracardiac electrogram data.

80. The method of any one of claims 70 to 79, wherein indicating on the image the identification of the heart tissue comprises coloring the position of the heart tissue in the image by a color predetermined for that heart tissue.

81. A method of guiding a structural heart disease intervention, comprising:
accessing a structural representation of a heart;
accessing electrophysiological measurements indicating electrical activity of tissue of the heart;
associating the electrophysiological measurements to locations in the structural representation of the heart corresponding to locations at which the measurements were recorded;
selecting a location for attachment of a device configured to provide structural heart disease intervention, based on the structural representation, the electrophysiological measurements, and their locations in the structural representation; and
presenting an image of the structural representation of the heart wherein the selected location is marked.

82. A method of guiding a structural heart disease intervention, comprising: accessing electrophysiological (EP) measurements of the heart measured from a specified location; and guiding the structural heart disease intervention based on the accessed EP measurements.

83. The method of claim 82, wherein guiding the structural heart disease intervention comprises indicating on an image of a portion of the heart a current location of an implant for use in the intervention, the specified location, and the accessed EP measurements.

84. The method of claim 82, wherein guiding the structural heart disease intervention comprises guiding an intervention tool to a location within the heart to be treated with the structural heart disease intervention.
